SEQUENCE LISTING (32 sequences)

SEQ ID NO: 1 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 2 - protein, 330 aa, Homo sapiens [sequence omitted]

SEQ ID NO: 3 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 6 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 9 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 20 bp, Artificial Sequence
  Description of Artificial Sequence Synthetic primer
gctgcctgaagaggaaattg

SEQ ID NO: 11 - DNA, 20 bp, Artificial Sequence
  Description of Artificial Sequence Synthetic primer
gcccagatgaagagagcttg

SEQ ID NO: 12 - DNA, 20 bp, Artificial Sequence
  Description of Artificial Sequence Synthetic primer
cttcagccatcgcaaggagt

SEQ ID NO: 13 - DNA, 20 bp, Artificial Sequence
  Description of Artificial Sequence Synthetic primer
gtttctccctgaaactcggc

SEQ ID NO: 14 - DNA, 25 bp, Artificial Sequence
  Description of Artificial Sequence Synthetic primer
acaagaagtaccaaacattgacaca

SEQ ID NO: 15 - DNA, 25 bp, Artificial Sequence
  Description of Artificial Sequence Synthetic primer
ttaactgcgtttaagacaaaattcc

SEQ ID NO: 16 - DNA, 20 bp, Artificial Sequence
  Description of Artificial Sequence Synthetic primer
tcccaggcctgaagacaatc

SEQ ID NO: 17 - DNA, 21 bp, Artificial Sequence
  Description of Artificial Sequence Synthetic primer
gctcgtaaggtcagctggtat

SEQ ID NO: 18 - DNA, 20 bp, Artificial Sequence
  Description of Artificial Sequence Synthetic primer
accagcagacactccgggct

SEQ ID NO: 19 - DNA, 20 bp, Artificial Sequence
  Description of Artificial Sequence Synthetic primer
acgtcttggctgggtgctcg

SEQ ID NO: 20 - DNA, 22 bp, Artificial Sequence
  Description of Artificial Sequence Synthetic primer
gagatacagactgctggcatag

SEQ ID NO: 21 - DNA, 22 bp, Artificial Sequence
  Description of Artificial Sequence Synthetic primer
gtagatgtaagtacgggcacag

SEQ ID NO: 22 - DNA, 24 bp, Artificial Sequence
  Description of Artificial Sequence Synthetic primer
ttatgtacccggcgatggcaccgg

SEQ ID NO: 23 - DNA, 22 bp, Artificial Sequence
  Description of Artificial Sequence Synthetic primer
tgctcgcaaccagttgccgatg

SEQ ID NO: 24 - DNA, 20 bp, Artificial Sequence
  Description of Artificial Sequence Synthetic primer
ggaacacctacttccggtcc

SEQ ID NO: 25 - DNA, 22 bp, Artificial Sequence
  Description of Artificial Sequence Synthetic primer
gggaaagaggagttgggacaat

SEQ ID NO: 26 - DNA, 22 bp, Artificial Sequence
  Description of Artificial Sequence Synthetic primer
tgtagaccatgtagtgaggtca

SEQ ID NO: 27 - DNA, 21 bp, Artificial Sequence
  Description of Artificial Sequence Synthetic primer
aggtcggtgtgaacggatttg

SEQ ID NO: 28 - protein, 400 aa, Homo sapiens
MVRPLNPRPLPPVVLMLLLLLPPSPLPLAAGDPLPTESRLMNSCLQARRKCQADPTCSAAYHHLDSCTSSISTPLPSEEPSVPADCLEAAQQLRNSSLIGCMCHRRMKNQVACLDIYWTVHRARSLGNYELDVSPYEDTVTSKPWKMNLSKLNMLKPDSDLCLKFAMLCTLNDKCDRLRKAYGEACSGPHCQRHVCLRQLLTFFEKAAEPHAQGLLLCPCAPNDRGCGERRRNTIAPNCALPPVAPNCLELRRLCFSDPLCRSRLVDFQTHCHPMDILGTCATEQSRCLRAYLGLIGTAMTPNFVSNVNTSVALSCTCRGSGNLQEECEMLEGFFSHNPCLTEAIAAKMRFHSQLFSQDWPHPTFAVMAHQNENPAVRPQPWVPSLFSCTLPLILLLSLW

SEQ ID NO: 29 - protein, 397 aa, Mus musculus
MGLSWSPRPPLLMILLLVLSLWLPLGAGNSLATENRFVNSCTQARKKCEANPACKAAYQHLGSCTSSLSRPLPLEESAMSADCLEAAEQLRNSSLIDCRCHRRMKHQATCLDIYWTVHPARSLGDYELDVSPYEDTVTSKPWKMNLSKLNMLKPDSDLCLKFAMLCTLHDKCDRLRKAYGEACSGIRCQRHLCLAQLRSFFEKAAESHAQGLLLCPCAPEDAGCGERRRNTIAPSCALPSVTPNCLDLRSFCRADPLCRSRLMDFQTHCHPMDILGTCATEQSRCLRAYLGLIGTAMTPNFISKVNTTVALSCTCRGSGNLQDECEQLERSFSQNPCLVEAIAAKMRFHRQLFSQDWADSTFSVVQQQNSNPALRLQPRLPILSFSILPLILLQTLW

SEQ ID NO: 30 - protein, 228 aa, Homo sapiens
MELGLGGLSTLSHCPWPRQQAPLGLSAQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 31 - protein, 224 aa, Mus musculus
MELGLAEPTALSHCLRPRWQSAWWPTLAVLALLSCVTEASLDPMSRSPAARDGPSPVLAPPTDHLPGGHTAHLCSERTLRPPPQSPQPAPPPPGPALQSPPAALRGARAARAGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 32 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

(19) United States
(12) Patent Application Publication
(10) Pub. No.: US 2017/0281756 A1
(43) Pub. Date: Oct. 5, 2017

(54) COMPOSITIONS AND METHODS TO SELECTIVELY BLOCK PAIN INDUCED BY COLD

(71) Applicant: University of Southern California, Los Angeles, CA (US)

(72) Inventor: David D. McKemy, Los Angeles, CA (US)

(21) Appl. No.: 15/443,475

(22) Filed: Feb. 27, 2017

Related U.S. Application Data
(60) Provisional application No. 62/299,983, filed on Feb. 25, 2016.

(51) Int. Cl. A61K 39/395; C07K 14/705; A61K 51/10

(52) U.S. Cl. CPC A61K 39/395; A61K 51/10; C07K 14/705; A61K 39/00

(57) ABSTRACT

Tissue injury prompts the release of a number of proalgesic molecules that induce acute and chronic pain by sensitizing pain-sensing neurons (nociceptors) to heat and mechanical stimuli. In contrast, many proalgesics have no effect on cold sensitivity, or can inhibit cold-sensitive neurons and diminish cooling-mediated pain-relief (analgesia). Nonetheless, cold pain (allodynia) is prevalent in many inflammatory and neuropathic pain settings, with little known of the mechanisms promoting pain versus those dampening analgesia. This disclosure establishes provides methods and compositions to treat cold allodynia induced by inflammation, nerve injury, and chemotherapeutics. One such therapy is the administration of antibodies against the GFRα3 ligand, artemin.

### CROSS-REFERENCE TO RELATED APPLICATION

This application claims priority under 35 U.S.C. §119(e) to U.S. Provisional Application No. 62/299,983, filed Feb. 25, 2016, the content of which is incorporated herein by reference in its entirety.

### STATEMENT OF GOVERNMENT SUPPORT

This invention was made with government support under Grant Nos. R01NS087542 and R21NS071364 awarded by National Institutes of Health (NIH). The government has certain rights in the invention.

### BACKGROUND

This disclosure references various publications, patents and published patent specifications by an identifying citation or an Arabic number. The full citations for the disclosures referenced by an Arabic number are found immediately preceding the claims. The disclosures of these publications, patents and published patent specifications are hereby incorporated by reference into the present disclosure in their entirety to more fully describe the state of the art to which this invention pertains.

When pain continues past its usefulness as a warning of potential tissue damage, it becomes a debilitating condition for which few viable treatments are currently available. The result can be an exacerbation of pain in response to both innocuous (allodynia) and noxious (hyperalgesia) stimuli (1). For example, pain felt with normally mild cooling (cold allodynia) occurs in many pathological conditions such as fibromyalgia, multiple sclerosis, stroke, and chemotherapeutic-induced polyneuropathy, yet what underlies this specific form of pain at the cellular or molecular level is largely unknown (2-5). Pain-sensing afferent neurons (nociceptors) are sensitized during injury or disease, in part, by a vast array of proalgesic compounds, termed the “inflammatory soup” (e.g. neurotrophic factors, protons, bradykinin, prostaglandins, ATP) (1). These substances are released locally at the site of injury by infiltrating immune cells such as macrophages, neutrophils, and T cells, as well as by resident cells including keratinocytes and mast cells (6), and either directly activate sensory receptors, or sensitize them to subsequent stimuli (7). Moreover, prolonged inflammation can lead to central sensitization (in the spinal cord and brain) and bring about long-lasting chronic pain that persists after acute inflammation has been resolved. Thus, a better understanding of the molecules involved in neuroinflammation may lead to therapeutic options for acute and chronic pain.

Tissue injury prompts the release of a number of proalgesic molecules that induce acute and chronic pain by sensitizing painsensing neurons (nociceptors) to heat and mechanical stimuli. In contrast, many proalgesics have no effect on cold sensitivity, or can inhibit cold-sensitive neurons and diminish cooling-mediated painrelief (analgesia). Nonetheless, cold pain (allodynia) is prevalent in many inflammatory and neuropathic pain settings, with little known of the mechanisms promoting pain versus those dampening analgesia.

This disclosure provide therapies and methods to treat these unmet needs.

### SUMMARY

The primary cold receptor in the peripheral nervous system is the transient receptor potential melastatin 8 (TRPM8) channel, which is expressed in a small subset of sensory neurons (8, 9). TRPM8 channels mediate the perception of both acute noxious and innocuous cold, and are key in cold allodynia after injury, as well as paradoxically mediate cooling-induced analgesia (10-15). The channel's involvement in cold allodynia is unclear as, unlike the broad range of proalgesics that sensitize the heat-gated capsaicin receptor TRPV1 to bring about thermal hyperalgesia (7, 16), cellular mechanisms that lead to sensitization of TRPM8 have not been identified (17). Conversely, proalgesics such as bradykinin, histamine, and prostaglandin E2 inhibit TRPM8 channel activity via a mechanism involving their respective G-protein coupled receptors (GPCR) and direct channel inhibition by the G-protein subunit Gaq (18, 19). This paradoxical inhibition of cold-sensitive nerves is considered a mechanism that would dampen cooling analgesia, thereby potentiating heat and mechanical hyperalgesia.

The channel TRPA1 has also been implicated in cold pain. Cold allodynia induced by inflammation was diminished by TRPA1 antagonism (20, 21), and an endogenous TRPA1 agonist promoted cold hypersensitivity in wildtype but not Trpa1−/− mice (21). As cold hypersensitivity is also reduced in Trpm8−/− mice (12, 14, 22), it remains to be determined how TRPA1 activation leads to cold hypersensitivity and if this process works through TRPM8 or other mechanisms. Nonetheless, cold allodynia is a persistent outcome of both inflammatory and neuropathic pain and elucidating the molecular mechanisms involved is critical for any therapeutic intervention.

Of the range of proalgesics known to promote pain, only nerve growth factor (NGF) and the glial cell-line derived neurotrophic factor (GDNF) family ligand (GFL) artemin have been shown to lead to cold hypersensitivity (18, 19, 23, 24). Each are major components of the inflammatory soup and produce nociceptor sensitization and pain via their cognate cell surface receptors. NGF, the classical proalgesic neurotrophic factor, leads to thermal and mechanical sensitization directly through its receptor tyrosine kinase TrkA expressed on nociceptors, and indirectly via the activation of peripheral cells (25). GDNF family of receptors (GFRαs) are typically coupled to the receptor tyrosine kinase Ret (1). However, GFRαs are more widely expressed than Ret and Ret-independent GFL-induced neuronal sensitization has been reported, suggesting that these receptor may signal through additional transmembrane proteins (26-29).

Both artemin and NGF are upregulated in conditions of inflammation or neuropathy, and each can induce thermal hypersensitivity (23, 25, 30-32). It has been previously shown that injection of artemin or NGF in the skin induces a robust and transient cold allodynia in mice (23). Moreover, the receptor for artemin, GFRα3, is co-expressed with TRPA1, and in a subpopulation of TRPM8 neurons that are almost exclusively TRPV1-positive. Artemin has been shown to inhibit TRPA1 activity in sensory neurons (33), results consistent with Applicant's findings that artemin-induced1′ cold allodynia is TRPM8-dependent (23).

Here, it is shown that cold allodynia induced by inflammation, nerve injury, or chemotherapeutics is completely abolished in mice null for GFRα3 (Gfrα3−/−). In contrast, heat and mechanical hyperalgesia are unaltered in Gfrα3−/− mice, indicating that GFRα3 has a limited role in pain associated with these sensory modalities, but predominates the potentiation of cold sensitivity after injury. This specificity strongly suggests that therapeutic interventions into cold allodynia should focus on artemin/GFRα3 signaling. Indeed it is shown that pathological cold pain alone is ameliorated in animals treated with artemin-neutralizing antibodies. These results show that cold allodynia is mediated exclusively by artemin-GFRα3 signaling, and that blocking this pathway is a viable treatment option for cold pain.

There are few effective treatments for chronic cold pain induced by tissue damage, nerve injury, or chemotherapeutic polyneuropathies. Of the vast array of pain-producing proalgesics inducing heat and mechanical pain, only the neurotrophic factors artemin and nerve growth factor (NGF) produce cold sensitivity. Here it is shown that the specific artemin receptor, GFRα3, is absolutely required for injury induced cold pain, whereas heat and mechanical pain are intact in mice lacking this receptor. It is also shown that pre-existing cold pain, or that induced by NGF, is completely alleviated with systemic administration of artemin-neutralizing antibodies. These results demonstrate that pathological cold pain is mediated exclusively by artemin-GFRα3 signaling, suggesting a specific transduction pathway that can be targeted selectively to treat cold pain.

Thus, in one aspect, provided herein is a method to treat pre-exising cold pain in a subject in need thereof, comprising, or alternatively consisting essentially of, or yet further consisting of, administering to the subject an effective amount of an agent that interferes with GFRα3 signaling in a cell or a tissue expressing the GFRα3 receptor. In one aspect, the pre-existing cold pain is induced by NGF in the subject. In one aspect, the subject is an animal, e.g., the subject is a human.

Also provided herein is a method to inhibit or reduce pre-exising cold pain in a subject in need thereof, comprising, or alternatively consisting essentially of, or yet further consisting of, administering to the subject an effective amount of an agent that interferes with GFRα3 signaling in a cell or a tissue expressing the GFRα3 receptor. In one aspect, the pre-existing cold pain is induced by NGF in the subject. In one aspect, the subject is an animal, e.g., the subject is a human.

Also provided herein is a method for the treatment of a subject a disease or condition that relates to GFRα3 receptor signaling in a cell or tissue in a subject in need thereof, comprising, or alternatively consisting essentially of, or yet further consisting of, administering to the subject an effective amount of an agent that interferes with GFRα3 signaling in the cell or the tissue expressing the GFRα3 receptor. In a further aspect, the agent interferes with artemin-GFRα3 signaling in the cell. In one aspect, the pre-existing cold pain is induced by NGF in the subject. In one aspect, the subject is an animal, e.g., the subject is a human.

In one aspect, the agent that is administered to the subject comprises, or alternatively consists of, or consists essentially of, an artemin neutralizing antibody or a fragment, derivative or variant of the artemin neutralizing antibody. In another aspect, the agent that is administered to the subject comprises, or alternatively consists of, or consists essentially of, a NGF neutralizing antibody or a fragment, derivative or variant of the NGFneutralizing antibody. In one aspect, the antibody is a human or an humanized antibody. A non-limiting example of such an agent comprises, or alternatively consists essentially of, or yet further consists of, the CDR of monoclonal antibody 1085 (mAb 1085), or an equivalent of the CDR of mAb 1085.

Also provided is a method to identify a therapeutic agent to treat and/or inhibit, and/or reduce pre-existing cold pain and/or that is induced by NGF, or a condition related to GFRα3 signaling in a subject in need thereof, comprising contacting a cell that expresses an GFRα3 receptor with a test agent and assaying for interference with GFRα3 signaling in the cell, wherein an agent that interferes with GFRα3 receptor signaling is a potential therapeutic agent and an agent that does not interfere with GFRα3 receptor signaling is not a potential therapeutic agent. In one aspect, the cell is an animal cell, e.g., a mammalian cell such as a human cell.

### BRIEF DESCRIPTION OF THE DRAWINGS

FIGS. 1A-1E show Gfrα3−/− mice display normal acute cold, heat and mechanosensory behaviors. Withdrawal latencies in response to cold (A) and heat (C) stimuli were similar in naïve adult (8-14 weeks of age) Gfrα3−/− and wildtype mice (p>0.05, n=5-12). (B) Paw withdrawal threshold forces did not differ between genotypes in the electronic von Frey assay (p>0.05, n=5-10). Gfrα3−/− mice injected with artemin (20 μg; p>0.05 for all time points, n=7) showed no change in cold sensitivity (E) compared to vehicle-injected mice (D) in the cold plantar assay (**p<0.01, ***p<0.001, n=7).

FIGS. 2A-2C show GFRα3 is required for cold allodynia induced by inflammation, nerve injury, and chemotherapy polyneuropathy. (A) Decreased cold-evoked hindpaw withdrawal latencies were observed in wildtype but not Gfrα3−/− mice in the cold plantar assay 2-days after an intraplantar injection of CFA (**p<0.01, n=7-9). Post-CFA latencies for Gfrα3−/− mice were not statistically different (p>0.05) than basal. (B) Similarly, cold allodynia observed in the ipsilateral hindpaw in wildtype mice (***p<0.001) after chronic constriction injury (CCI) was absent in Gfrα3−/− mice with post-injury withdrawal latencies identical to pre-injury times (p>0.05, n=6-7). (C) Oxaliplatin-induced decreases in withdrawal latencies to cold observed in wildtype controls (***p<0.001) were absent in Gfrα3−/− mice, with response times the same as pre-injection times for both genotypes (p>0.05, n=11-12). Increased acetone-cooling evoked response score was observed in wildtype but not Gfrα3−/− mice after an intraplantar injection of CFA (a, **p<0.01, n=8), after chronic constriction injury (CCI) of the sciatic nerve (b, **p<0.01, n=7), and in oxaliplatin polyneuropathy (c, **p<0.01, n=9). Post-injury scores for Gfrα3−/− mice were not statistically different (p>0.05) between ipsilateral and contralateral hind paws (CFA and CCI) or versus basal (oxalplatin).

FIGS. 3A-3E show heat or mechanical hyperalgesia are not dependent on GFRα3. Both wildtype and Gfrα3−/− mice exhibit reduced threshold forces inducing a paw withdrawal 3-days after unilateral CFA injection (A) or 7-days after CCI surgery (B) (***p<0.001, ipsi vs. contra, n=6-7). Similarly, cold allodynia observed 7-days after a single injection of oxaliplatin (C) was robust in both genotypes (***p<0.001, n=6-8), as was heat hyperalgesia (***p<0.001, ipsi vs. contra, n=7-8) 3-days after the induction of inflammation (E) or 7-days after nerve injury (D).

FIGS. 4A-4F show expression of somatosensory markers are unaltered in the absence of GFRα3. Representative images of triple labeled DRG sections from both adult wildtype (top rows) and Gfrα3−/− (bottom rows) mice. In all images TRPM8 (light grey) and GFRα3 (dark grey) expression is compared to a difference marker in grey. (A) TRPV1 labeling in DRGs and their overlap with TRPM8 (light grey) and showed no differences in thermosensory TRP channel expression between genotypes. Similarly, expression of the peptide CGRP (B), the non-peptidergic marker IB4 (C), the A-fiber marker NF200 (D), and the C-fiber marker peripherin (E) were similar in wildtype and Gfrα3−/− mice. Arrowheads indicate triple-labeled neurons (wildtype) or double-labeled neurons (Gfrα3−/−), whereas arrows indicate TRPM8 cells that are not positive for either marker. (F) Quantification of immunohistochemical labeling of various sensory neuron makers shows similar proportions in Gfrα3−/− and wildtype controls DRGs. Percentages of total DRG neurons that were positive for each marker are similar in Gfrα3−/− tissue compared to wildtype tissue (p>0.05, n=15-32 sections from at least 3 mice).

FIGS. 5A-5E shows artemin-neutralization selectively attenuates cold hypersensitivity. (A) Inflammation-induced (CFA) cold hypersensitivity was attenuated in wildtype mice 4 hrs after subcutaneous injection of an anti-artemin antibody (MAB1085, 10 mg/kg, p>0.05 ipsilateral versus contralateral, n=6-7) compared to mice injected with an IgG2A isotype control (10 mg/kg, **p<0.01). Similarly, chemotherapeutic-induced cold pain (B) was attenuated after MAB1085 injection (p>0.05 pre-oxaliplatin versus post-MAB1085) and significantly different from control-injected mice (**p<0.01). Conversely, inflammatory mechanical hyperalgesia (C) was unaffected by MAB1085 treatment (p>0.05 ipsilateral control versus ipsilateral MAB1085 injected, ***p<0.001 ipsilateral versus contralateral for both treatments, n=5-8). (D) Chemotherapeutic-induced mechanical hyperalgesia was also unaffected by treatment with MAB1085 (p>0.05 post-MAB1085 versus control, **p<0.01 pre-oxaliplatin vs. post-MAB1085, n=5-6). (E) Inflammatory thermal hyperalgesia was unaffected by MAB1085 treatment (p>0.05 ipsilateral control versus ipsilateral MAB1085 injected, ***p<0.001 ipsilateral versus contralateral for both treatments, n=6).

FIGS. 6A-6D show NGF-induced cold allodynia is GFRα3-dependent. (A) Wildtype mice exhibit cold allodynia 1 hr, but not 3 hr, after intraplantar NGF injections (**p<0.01, p>0.05, n=9-11), whereas Gfrα3−/− mice (B) showed no change in cold sensitivity compared to vehicle-injected mice in the cold plantar assay (p>0.05, n=9-11). Both wildtype (C) and Gfrα3−/− mice (D) displayed robust heat hyperalgesia 1 and 3 hrs after NGF administration (*p<0.05, **p<0.01, ***p<0.001, n=6).

FIGS. 7A-7B show artemin-neutralization blocks NGF-induced cold allodynia. (A) NGF-induced cold allodynia was attenuated in wildtype mice given a systemic injection of MAB1085 (10 mg/k, p>0.05 pre vs. post-NGF and vs. veh-injected mice, n=4) 1 hr prior to intraplantar NGF injection. Control mice show robust cold allodynia after NGF injection (***p>0.001, pre vs. post-NGF and vs. veh-injected, n=4). (B) NGF-induced heat hyperalgesia was unaffected by MAB1085 treatment and similar to controls (***p>0.001, pre vs. post-NGF and vs. veh-injected, n=4).

FIG. 8 shows artemin-induced cold allodynia is absent in Gfrα3^{-/-} mice. Gfrα3^{-/-} mice injected with artemin (intraplantar; 20 μg) showed no change in cold sensitivity when compared to vehicle injection in the evaporative cooling assay (p>0.05 for all time points, n=5-6).

FIGS. 9A-9B show expression of genes involved in cold transduction is not dependent on GFRα3. Complementary DNA (cDNA) purified from adult dorsal root (A) and trigeminal ganglia (B) was analyzed by qPCR, showing that transcript levels of a number of genes involved in somatosensory signaling are similar between Gfrα3^{-/-} and wildtype tissues (n=4 mice).

FIG. 10 shows artemin-neutralization attenuates cold hypersensitivity in the evaporative cooling assay. Inflammation-induced (CFA) cold hypersensitivity was attenuated in wildtype mice 4 hrs after subcutaneous injection of an anti-artemin antibody (MAB1085, 10 mg/kg, p>0.05 ipsilateral versus contralateral, n=8) compared to mice injected with an IgG2A isotype control (10 mg/kg, **p<0.01).

### DETAILED DESCRIPTION

### Definitions

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to proteins, polypeptides, cells, nucleic acids, such as DNA or RNA, refers to molecules separated from other proteins, polypeptides, cells, nucleic acids, such as DNA or RNA, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together. A recombinant polynucleotide is a polynucleotide created or replicated using techniques (chemical or using host cells) other than by a cell in its native environment.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

“Cells,” “host cells” or “recombinant host cells” are terms used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Pat. Nos. 4,683,195 and 4,683,202 and Innis et al. (1990) Mol. Cell Biol. 10(11):5977-5982 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype” refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on May 21, 2008. Biologically equivalent polynucleotides are those having the above-noted specified percent homology and encoding a polypeptide having the same or similar biological activity.

As used herein, the term “biological equivalent thereof” is intended to be synonymous with “equivalent thereof” when referring to a reference protein, antibody, polypeptide, polynucleotide or nucleic acid, and intends those having minimal homology while still maintaining desired structure or functionality. Unless specifically recited herein, it is contemplated that any nucleic acid, polynucleotide, polypeptide, protein or antibody mentioned herein also includes equivalents thereof. For example, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide, antibody or nucleic acid.

In one aspect, the term “equivalent” as it refers to polypeptides, proteins, or polynucleotides refers to polypeptides, proteins, or polynucleotides, respectively having a sequence having a certain degree of homology or identity with the reference sequence of the polypeptides, proteins, or polynucleotides (or complement thereof when referring to polynucleotides). A homolog of a double stranded nucleic acid is intended to include nucleic acids having a nucleotide sequence that has a certain degree of homology with or with the complement thereof. In one aspect, homologs of nucleic acids are capable of hybridizing to the nucleic acid or complement thereof. In one aspect, an equivalent has at least 70%, or at least 75% or at least 80%, or at least 85%, or at least 90%, or at least 95%, or at least 97%, or at least 98%, sequence identity to the reference polynucleotide or polypeptide.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

As used herein, the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

As used herein, a “vector” is a vehicle for transferring genetic material into a cell. Examples of such include, but are not limited to plasmids and viral vectors. A viral vector is a virus that has been modified to transduct genetic material into a cell. A plasmid vector is made by splicing a DNA construct into a plasmid. As is apparent to those of skill in the art, the appropriate regulatory elements are included in the vectors to guide replication and/or expression of the genetic material in the selected host cell.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral Vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmic vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacteria produces proteins to confer its antibiotic resistance, it can also be induced to produce large amounts of proteins from the inserted gene. This is a cheap and easy way of mass-producing a gene or the protein it then codes for.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells. Non-limiting examples include simian, bovine, ovine, porcine, murine, rats, canine, equine, feline, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to prokaryotic Cyanobacteria, bacillus bacteria, E. coli bacterium, and Salmonella bacterium.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′ —OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optionall contain detectable labels and are exemplified and described herein.

As used herein, the term “detectable label” intends a directly or indirectly detectable compound or composition (other than a naturally occurring polynucleotide in its natural environment) that is conjugated directly or indirectly to the composition to be detected, e.g., polynucleotide or protein such as an antibody so as to generate an artificial, non-naturally occuring “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are known in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarry system that can dispense from 12 to 64 spots onto multiple glass slides (Intelligent Bio-Instruments); Molecular Biology Workstation and NanoChip (Nanogen, Inc.); a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarrays” are also described in U.S. Patent Publication Nos.: 2007/0111322; 2007/0099198; 2007/0084997; 2007/0059769 and 2007/0059765 and U.S. Pat. Nos. 7,138,506; 7,070,740 and 6,989,267.

In one aspect, “gene chips” or “microarrays” containing probes or primers homologous to a polynucleotide described herein are prepared. A suitable sample is obtained from the patient, extraction of genomic DNA, RNA, protein or any combination thereof is conducted and amplified if necessary. The sample is contacted to the gene chip or microarray panel under conditions suitable for hybridization of the gene(s) or gene product(s) of interest to the probe(s) or primer(s) contained on the gene chip or microarray. The probes or primers may be detectably labeled thereby identifying the sequence(s) of interest. Alternatively, a chemical or biological reaction may be used to identify the probes or primers which hybridized with the DNA or RNA of the gene(s) of interest. The genotypes or phenotype of the patient is then determined with the aid of the aforementioned apparatus and methods.

It is to be inferred without explicit recitation and unless otherwise intended, that when the present technology relates to a polypeptide, protein, polynucleotide or antibody, an equivalent or a biologically equivalent of such is intended within the scope of the present technology.

As used herein, the “administration” of an agent or drug to a subject or subject includes any route of introducing or delivering to a subject a compound to perform its intended function. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, vaginal, nasal administration, injection, topical application and by suppository. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

Administration can be effected in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician.

As used herein, the term “animal” refers to living multi-cellular vertebrate organisms, a category that includes, for example, mammals and birds. The term “mammal” includes both human and non-human mammals. Similarly, the term “subject” or “patient” includes both human and veterinary subjects, for example, humans, non-human primates, dogs, cats, sheep, mice, horses, and cows.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, “antibody” refers to a polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

The terms “polyclonal antibody” or “polyclonal antibody composition” as used herein refer to a preparation of antibodies that are derived from different B-cell lines. A polyclonal antibody composition comprises a mixture of immunoglobulin molecules secreted against a specific antigen, recognizing the same or different epitopes of the antigen, or against different antigens.

The terms “monoclonal antibody” or “monoclonal antibody composition” as used herein refer to a preparation of antibody molecules of single molecular composition. A monoclonal antibody composition displays a single binding specificity and affinity for a particular epitope.

A “monoclonal antibody mixture” or an “oligoclonal cocktail” refers to a mixture or combination of multiple monoclonal antibodies, each of which monoclonal antibodies can specifically recognize and bind the same antigen, the same or different epitopes of the antigen, or different antigens.

Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds artemin or its receptor will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and C_{H}, domains; a F(ab′)₂ fragment, a bivalent fragment comprising two Fab fragments linked by a disulfide bridge at the hinge region; a F_{d} fragment consisting of the V_{H} and C_{H}, domains; a Fᵥ fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fᵥ (scFᵥ)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

“Antibody fragments” or “antigen binding fragments” include proteolytic antibody fragments (such as F(ab′)₂ fragments, Fab′ fragments, Fab′-SH fragments and Fab fragments as are known in the art), recombinant antibody fragments (such as sFᵥ fragments, dsFᵥ fragments, bispecific sFᵥ fragments, bispecific dsFᵥ fragments, F(ab)′₂ fragments, single chain Fv proteins (“scFᵥ”), disulfide stabilized Fᵥ proteins (“dsFᵥ”), diabodies, and triabodies (as are known in the art), and camelid antibodies (see, for example, U.S. Pat. Nos. 6,015,695; 6,005,079; 5,874,541; 5,840,526; 5,800,988; and 5,759,808). An scFᵥ protein is a fusion protein in which a light chain variable region of an immunoglobulin and a heavy chain variable region of an immunoglobulin are bound by a linker, while in dsFᵥs, the chains have been mutated to introduce a disulfide bond to stabilize the association of the chains.

As used herein, the term “antibody derivative” is intended to encompass molecules that bind an epitope as defined herein and which are modifications or derivatives of an isolated neutralizing antibody of the present technology. Derivatives include, but are not limited to, for example, bispecific, heterospecific, tri specific, tetraspecific, multispecific antibodies, diabodies, chimeric, recombinant and humanized. As used herein, the term “bispecific molecule” is intended to include any agent, e.g., a protein, peptide, or protein or peptide complex, which has two different binding specificities. As used herein, the term “multispecific molecule” or “heterospecific molecule” is intended to include any agent, e.g., a protein, peptide, or protein or peptide complex, which has more than two different binding specificities. As used herein, the term “heteroantibodies” refers to two or more antibodies, antibody binding fragments (e.g., Fab), derivatives thereof, or antigen binding regions linked together, at least two of which have different specificities.

The term “antibody variant” is intended to include antibodies produced in a species other than a rabbit. It also includes antibodies containing post-translational modifications to the linear polypeptide sequence of the antibody or fragment. It further encompasses fully human antibodies.

In one aspect, the term “equivalent” or “biological equivalent” of an antibody means the ability of the antibody to selectively bind its epitope protein or fragment thereof as measured by ELISA, IHC or other suitable methods. Biologically equivalent antibodies include, but are not limited to, those antibodies, peptides, antibody fragments, antibody variant, antibody derivative and antibody mimetics that bind to the same epitope as the reference antibody. The skilled artisan can prepare an antibody functionally equivalent to the antibodies of the present disclosure by introducing appropriate mutations into the antibody using site-directed mutagenesis (Hashimoto-Gotoh, T. et al., Gene 152, 271-275 (1995); Zoller & Smith, Methods Enzymol. 100, 468-500 (1983); Kramer, W. et al., Nucleic Acids Res. 12, 9441-9456 (1984); Kramer W. & Fritz H J., Methods. Enzymol. 154, 350-367 (1987); Kunkel, T A., Proc Natl Acad Sci USA. 82, 488-492 (1985); and Kunkel Methods Enzymol. 85, 2763-2766 (1988)).

Antibodies that are functionally equivalent to the antibodies of the present disclosure and comprise an amino acid sequence comprising mutation of one or more amino acids in the amino acid sequence of an antibody disclosed herein are also included in the antibodies of the present technology. In such mutants, the number of amino acids that are mutated is generally 50 amino acids or less, preferably 30 or less, and more preferably 10 or less (for example, 5 amino acids or less). An amino acid residue is preferably mutated into one that conserves the properties of the amino acid side chain. For example, based on their side chain properties, amino acids are classified into:

hydrophobic amino acids (A, I, L, M, F, P, W, Y, and V);

hydrophilic amino acids (R, D, N, C, E, Q, G, H, K, S, and T);

amino acids having aliphatic side-chains (G, A, V, L, I, and P);

amino acids having hydroxyl group-containing side-chains (S, T, and Y);

amino acids having sulfur atom-containing side-chains (C and M);

amino acids having carboxylic acid- and amide-containing side-chains (D, N, E, and Q);

base-containing side-chains (R, K, and H); and

amino acids having aromatic-containing side-chains (H, F, Y, and W).

(The letters within parentheses indicate one-letter amino acid codes).

As used herein, the term “neutralizing antibody” intends one that binds to its target and inhibits or diminishes the biological response by the target binding to its ligand.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens. In one aspect, the antigen is the GDNF family receptor alpha-3 protein, also known as the artemin receptor, or a fragment thereof. In another aspect, the antigen is the artemin protein or a fragment thereof. The sequence of the human GDNF protein is disclosed at NP_001487. A non-limiting example of an equivalent is the murine protein sequence found at NP_034410 (each incorporated herein by reference). The nucleic acid sequences encoding such are disclosed at NM_001496 and NM_010280, respecrtively. In a further aspect, the antigen is NGF,

Artemin, is also known as enovin or neublastin, is a protein, that in humans is encoded by the ARTN gene. The protein sequence of Artemin is disclosed at NP_001129687, and the murine protein (an example of an equivalent of the human protein is the murine protein, disclosed at NP_001271120. Polynucleotides encoding the proteins are disclosed at NM_001136215 and NM_001284191, respectively.

Nerve growth factor or “NGF” is a member of the NGF-beta family and encodes a secreted protein which homodimerizes and is incorporated into a larger complex. This protein has been shown to have nerve growth stimulating activity. The complex is reported to be involved in the regulation of growth and the differentiation of sympathetic and certain sensory neurons. The amino acid sequence for the human protein is known in the art (see UniProtKB-P01138; available at uniprot.org/uniprot/P01138) as well as homologs thereto. Purified and recombinant proteins are commercially available from a number of vendors, e.g., EMD Millipore; R&D Systems; and Enzo Life Sciences.

As used herein, a “disease or condition related to GFRα3 signalling” include, for example, one or more of: cold allodynia induced by inflammation, nerve injury, or chemotherapeutics (e.g., artemin, NGF or oxaliplatin), pathological cold pain, chronic cold pain induced by tissue damage, nerve injury, or chemotherapeutic polyneuropathies, injury-induced cold pain, cold allodynia that is localized at or near a site of injury or artemin exposure. Treatment can be monitored by any method known in the art, e.g., a reduction in pain or discomfort.

As used herein, “binding affinity” refers to the tendency of one molecule to bind (typically non-covalently) with another molecule, such as the tendency of a member of a specific binding pair for another member of a specific binding pair. A binding affinity can be measured as a binding constant, which binding affinity for a specific binding pair (such as an antibody/antigen pair) can be at least 1×10⁻⁵M, at least 1×10⁻⁶M, at least 1×10⁻⁷M, at least 1×10⁻⁸M, at least 1×10⁻⁹M, at least 1×10⁻¹⁰M, at least 1×10⁻¹¹ M or at least 1×10⁻¹² M. In one aspect, binding affinity is calculated by a modification of the Scatchard method described by Frankel et al., Mol. Immunol., 16:101-106, 1979. In another aspect, binding affinity is measured by an antigen/antibody dissociation rate. In yet another aspect, a high binding affinity is measured by a competition radioimmunoassay. In several examples, a high binding affinity for an antibody/antigen pair is at least about 1×10⁻⁸ M. In other aspects, a high binding affinity is at least about 1.5×10⁻⁸M, at least about 2.0×10⁻⁸M, at least about 2.5×10⁻⁸M, at least about 3.0×10⁻⁸M, at least about 3.5×10⁻⁸ M, at least about 4.0×10⁻⁸M, at least about 4.5×10⁻⁸ M, or at least about 5.0×10⁻⁸ M.

A “composition” typically intends a combination of the active agent, e.g., compound or composition, and a carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base that in one aspect, serves to stabilize the antibody in a formulation for storage. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, antimicrobial agents, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term carrier also includes typical pharmaceutically acceptable carriers, e.g., such as a phosphate buffered saline solution, water, and emulsions, such as an oil/water or water/oil emulsion, and various types of wetting agents. Examples of pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

As used herein, the term “biological sample” means sample material derived from or contacted by living cells. The term “biological sample” is intended to include tissues, cells and biological fluids isolated from a subject, as well as tissues, cells, and fluids present within a subject. Biological samples of the present disclosure include, e.g., but are not limited to, whole blood, plasma, semen, saliva, tears, urine, fecal material, sweat, buccal, skin, cerebrospinal fluid, and hair. Biological samples can also be obtained from biopsies of internal organs or from cancers. Biological samples can be obtained from subjects for diagnosis or research or can be obtained from healthy individuals, as controls or for basic research.

A “pharmaceutical composition” is intended to include the combination of an active agent with a carrier, inert or active, making the composition suitable for diagnostic or therapeutic use in vitro, in vivo or ex vivo.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a serum level commensurate with the concentrations found to be effective in vitro.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

A “control” is an alternative subject or sample used in an experiment for comparison purpose. A control can be “positive” or “negative”.

### Descriptive Embodiments

Provided herein are methods to treat and/or inhibit, and/or reduce pre-existing cold pain and/or a condition or disease that is induced by nerve growth factor (NGF) in a subject in need thereof. The methods comprise, or alternatively consist essentially of, or yet further consist of, administering to the subject of an effective amount of an agent that interferes with GFRα3 signaling in a cell or tissue expressing the GFRα3 receptor. In one aspect, the agent interferes with artemin-GFRα3 signaling in the cell. A non-limiting example of such an agent is an artemin neutralizing small molecule, an NGF neutralizing small molecule, an antibody or a fragment, derivative or variant of the artemin neutralizing antibody for example, an antibody that specifically binds to either or both of NGF or its receptor or an antibody or a fragment, derivative or variant of the NGF neutralizing antibody for example, an antibody that specifically binds to either or both of NGF or its receptor. Methods to generate such antibodies are known in the art and described herein. The antibody can be a polyclonal composition or a monoclonal antibody, a derivative or the antibody, a variant of the antibody, a diabody of the antibody, a bispecific antibody, or a fragment of any of each thereof. The antibody can be a species-ized antibody, e.g., a murine, a sheep, a goat, a rat, a canine, a human or an humanized antibody. One such antibody, that can be further modified as decribed herein, is the monoclonal antibody 1085 or an equivalent thereof. Hybridoma cell lines producing the antibodies are further provided herein.

In one aspect, the antibody comprises the CDR of monoclonal antibody 1085 (mAb 1085), or an equivalent of the CDR of mAb 1085.

Further provided are polynucleotides (DNA and/or RNA, e.g., mRNA) encoding the antibodies as described herein or fragments thereof which can be used as probes or primers. The polynucleotides can be inserted into an expression vector and a host cell for recombinant expression of the polypeptide produced by the expression system. The polynucleotide can futher comprise a detectable label, non-limiting examples of such are described herein. Non-limiting examples of vectors include plasmids, yeast, and viral vector systems. The polynucleotides are optionally opertatively linked to the appropriate expression or regulatory elements to drive expression within the vector or host cell. The host cell can be a prokaryotice or eukaryotic cell, e.g., a yeast cell, a murine cell, or a human cell. Accordingly, further provided is a method for expressing the antibody protein or a fragment thereof by growing the recombinant cell under conditions for the expression of the polynucleotide and optionally isolating the recombinant protein or polypepotide produced by the cell.

The agents, polynucleotides, antibodies, vectors, and/or host cells can be combined with a carrier, such as a pharmaceutically acceptable carrier for ease of administration. In one aspect, the polynucleotides or polypeptides can be combined with a solid support. As such, this disclosure also provides compositions comprising the agent and a carrier such as a pharmaceutically acceptable carrier or solid support. In addition the agents can further comprise a detectable agent or lable. In one aspect, the agent is combined with other therapeutic agents and/or suitable carriers for a particular dosing regimen.

The methods to treat and/or inhibit, and/or reduce pre-existing cold pain and/or a condition or related to or is induced by NGF can be practice on any suitable subject, e.g. an animal or a mammal, such as a human subject. When the animal is a non-human animal, the method can be used for preclinical testing of potential therapeutic agents identified by the methods disclosed herein.

An effective amount of the agent is administered to the agent by any appropriate means of administration, e.g., systemically or locally. The amount to be administered with vary with the subject, its age and general health and can be determined by the treating physician. The agents and methods can be combined with other suitable therapies or therapeutic agents as determined by the treating physician. Administration of the compositions or agent can be made by methods known in the art. In one aspect, the route of administration is intramuscular injection. In another aspect, the route of administration is intravenous injection. In another aspect, the route of administration is subcutaneous injection.

Administration of the agent or compositin can be composed of one dose, or a number of consecutive doses. The amount and frequency of dosage can be determined with methods known in the art, and will vary depending on factors such as the risk of continued risk of infection, half life of the antibody and toxicity of the formulation.

Administration of the agent or composition can be made at one site of the subject, or multiple sites of the subject. The amount of dosage and sites can be determined with methods known in the art. In one aspect, the administration is one or more intramuscular injection at the thigh or the arm of the subject. In another aspect, the administration is one or more intramuscular injection at the rear thigh of the subject. In a further aspect, administration is intravenously or orally.

Also provided herein is a method to identify a therapeutic agent to treat and/or inhibit, and/or reduce pre-existing cold pain and/or that induced by NGF, or that treats a condition related to GFRα3 receptor signaling in a subject in need thereof comprising contacting a cell or tissue that expresses an GFRα3 receptor with a test agent and assaying for interference with GFRα3 signaling in the cell, wherein an agent that interferes with GFRα3 signaling is a potential therapeutic agent and an agent that does not interfere with GFRα3 receptor signaling is not a potential therapeutic agent. In one aspect the cell is an animal or mammalian cell, e.g., a murine or a human cell.

Also provided herein is a method to identify a therapeutic agent to treat and/or inhibit, and/or reduce pre-existing cold pain and/or that induced by NGF, or that treats a condition related to NGF receptor signaling in a subject in need thereof comprising contacting a cell or tissue that expresses an NGF receptor with a test agent and assaying for interference with NGF signaling in the cell, wherein an agent that interferes with NGF receptor signaling is a potential therapeutic agent and an agent that does not interfere with NGF receptor signaling is not a potential therapeutic agent. In one aspect the cell is an animal or mammalian cell, e.g., a murine or a human cell.

Kits for administration of the agents or for use in the screeing method are further provided by this disclosure.

Antibody Compositions

In one aspect, this disclosure provides an agent that interferes with GFRα3 signaling in a cell or tissue expressing the GFRα3 receptor that has the therapeutic benefit as described herein. In one aspect, the agent interferes with artemin-GFRα3 signaling in the cell. A non-limiting example of such an agent is an artemin neutralizing small molecule or an antibody or a fragment, derivative or variant of the artemin neutralizing antibody for example, an antibody that specifically binds to either or both of artemin or its receptor (e.g., the GFRα3 receptor). The antibodies can be raised against the artemin protein or a fragment thereof and/or the GFRα3 receptor protein or a fragment thereof. Non-limiting examples of the amino acid sequences of such proteins are provided herein. A non-limiting example of an antibody is monoclonal antibody 1085 or an equivalent, or a fragment, derivative or variant thereof. The antibodies, once obtained can be further modified by methods known in the art or as described herein.

In another aspect, this disclosure provides an agent that interferes with NGF signaling in a cell or tissue expressing the NGF receptor that has the therapeutic benefit as described herein. A non-limiting example of such an agent is an NGF neutralizing small molecule or an antibody or a fragment, derivative or variant of the NGF neutralizing antibody for example, an antibody that specifically binds to either or both of NGF or its receptor. The antibodies can be raised against the NGF protein or a fragment thereof and/or the NGF receptor protein or a fragment thereof. Non-limiting examples of the amino acid sequences of such proteins are provided herein and are known in the art. The antibodies, once obtained can be further modified by methods known in the art or as described herein.

Methods to generate such antibodies are known in the art and described herein. The general structure of antibodies is known in the art and will only be briefly summarized here.

An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

Antibodies can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammal's serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antiben depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods known in the art some of which are described in U.S. Pat. Nos. 7,279,559; 7,119,179; 7,060,800; 6,709,659; 6,656,746; 6,322,788; 5,686,073; and 5,670,153.

Antibody derivatives of the present invention can also be prepared by delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

The antibodies or fragments thereof may be engineered, mutated or modified, e.g., peglyated, glycosylated, hinge-modified (see for example Filpula (2007) Biomol. Eng. 24(2): 201-15; Dall'Acqua et al (2006) J. Immunol. 177: 1129-38). Such changes can be also used to alter the PI of the antibody.

Antibody derivatives also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and references cited therein. Thus, antibodies can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

Antibodies of this disclosure include naturally purified products, products of chemical synthetic procedures, and products produced by recombinant techniques from a eukaryotic host, including, for example, yeast, higher plant, insect and mammalian cells, or alternatively from a prokaryotic host as described above. A number of antibody production systems are described in Birch & Radner (2006) Adv. Drug Delivery Rev. 58: 671-685.

In general, the CDR residues are directly and most substantially involved in influencing antigen binding. Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits. (1998) Advanced Drug Delivery Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this invention also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this invention can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in antibodies from other mammalian species. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed. Veneering can also be used to alter the PI of an antibody.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498. Veneering can also be used to alter the PI of an antibody.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Holliger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen).

The term “antibody derivative” further includes engineered antibody molecules, fragments and single domains such as scFv, dAbs, nanobodies, minibodies, Unibodies, and Affibodies (Holliger & Hudson (2005) Nature Biotech 23(9):1126-36; U.S. Patent Publication US 2006/0211088; PCT Publication WO2007/059782; U.S. Pat. No. 5,831,012).

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}-C_{H} 1-VH-C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

The term “antibody” also is intended to include antibodies of all isotypes. Particular isotypes of a monoclonal antibody can be prepared either directly by selecting from an initial fusion, or prepared secondarily, from a parental hybridoma secreting a monoclonal antibody of different isotype by using the sib selection technique to isolate class switch variants using the procedure described in Steplewski, et al. (1985) Proc. Natl. Acad. Sci. USA 82:8653 or Spira, et al. (1984) J. Immunol. Methods 74:307. Alternatively, recombinant DNA techniques may be used.

The isolation of other monoclonal antibodies with the specificity of the monoclonal antibodies described herein can also be accomplished by one of ordinary skill in the art by producing anti-idiotypic antibodies. Herlyn, et al. (1986) Science 232:100. An anti-idiotypic antibody is an antibody which recognizes unique determinants present on the monoclonal antibody of interest.

In some aspects of this invention, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

The antibodies also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

In some of the aspects of the antibodies provided herein, the antibody is soluble Fab fragment of the antibody.

In some of the aspects of the antibodies provided herein, the HC and LC variable domain sequences are components of the same polypeptide chain. In some of the aspects of the antibodies provided herein, the HC and LC variable domain sequences are components of different polypeptide chains.

In some of the aspects of the antibodies provided herein, the antibody is a full-length antibody.

In some of the aspects of the antibodies provided herein, the antibody is a monoclonal antibody.

In some of the aspects of the antibodies provided herein, the antibody is chimeric or humanized.

In some of the aspects of the antibodies provided herein, the antibody is selected from the group consisting of Fab, F(ab)′2, Fab′, scFᵥ, and Fᵥ.

In some of the aspects of the antibodies provided herein, the antibody comprises an Fc domain. In some of the aspects of the antibodies provided herein, the antibody is a rabbit antibody or a murine, or a rat, or a sheep antibody. In some of the aspects of the antibodies provided herein, the antibody is a human or humanized antibody or is non-immunogenic in a human.

In some of the aspects of the antibodies provided herein, the antibody comprises a human antibody framework region.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.

1) Amino acids with basic side chains: lysine, arginine, histidine.

2) Amino acids with acidic side chains: aspartic acid, glutamic acid

3) Amino acids with uncharged polar side chains: asparagine, glutamine, serine, threonine, tyrosine.

4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

The constant regions of antibodies may also be varied from those specifically disclosed for antibodies SP265. For example, antibodies may be provided with Fc regions of any isotype: IgA (IgA1, IgA2), IgD, IgE, IgG (IgG1, IgG2, IgG3, IgG4) or IgM. Non-limiting examples of constant region sequences include:

Human IgD constant region, Uniprot: P01880
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK
Human IgG1 constant region, Uniprot: P01857
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK
Human IgG3 constant region, Uniprot: P01860
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgM constant region, Uniprot: P01871
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY
Human IgG4 constant region, Uniprot: P01861
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human IgA1 constant region, Uniprot: P01876
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
Human IgA2 constant region, Uniprot: P01877
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRIVIAGKPTHVNVSVVMAEVDGTCY
Human Ig kappa constant region, Uniprot: P01834
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

In some aspects of the antibodies provided herein, the antibody contains structural modifications to facilitate rapid binding and cell uptake and/or slow release. In some aspects, the antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

The antibodies, fragments, and equivalents thereof can be combined with a detectable label and/or a carrier, e.g., a pharmaceutically acceptable carrier or other agents to provide a formulation for use and/or storage.

Further provided is an isolated polypeptide comprising, or alternatively consisting essentially of, or yet further consisting of, the proteins and fragments thereof as described herein that are useful to generate antibodies as well as isolated polynucleotides that encode them. In one aspect, the isolated polypeptides or polynucleotides further comprise a label and/or contiguous polypeptide sequences (e.g., keyhole limpet haemocyanin (KLH) carrier protein) or in the case of polynucleotides, polynucleotides encoding KLH, operatively coupled to polypeptide or polynucleotide. The polypeptides or polynucleotides can be combined with various carriers, e.g., phosphate buffered saline. Further provided are host cells, e.g., prokaryotic or eukaryotic cells, e.g., bacteria, yeast, mammalian (rat, simian, hamster, or human), comprising the isolated polypeptides or polynucleotides. The host cells can be combined with a carrier. In one aspect, the composition further comprises a cryoprotectant and/or a preservative. “Cryoprotectants” are known in the art and include without limitation, e.g., sucrose, trehalose, and glycerol. A cryoprotectant exhibiting low toxicity in biological systems is generally used. A “preservative” is a natural or synthetic chemical that is added to products such as foods, pharmaceuticals, paints, biological samples, wood, etc. to prevent decomposition by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Common antimicrobial preservatives include, benzalkonium chloride, benzoic acid, cholorohexidine, glycerin, phenol, potassium sorbate, thimerosal, sulfites (sulfur dioxide, sodium bisulfite, potassium hydrogen sulfite, etc.) and disodium EDTA. Other preservatives include those commonly used in patenteral proteins such as benzyl alcohol, phenol, m-cresol, chlorobutanol or methylparaben.

### Processes for Preparing Compositions

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain aspects, the antibodies of the present disclosure are polyclonal, i.e., a mixture of plural types of antibodies having different amino acid sequences. In one aspect, the polyclonal antibody comprises a mixture of plural types of antibodies having different CDRs. As such, a mixture of cells which produce different antibodies is cultured, and an antibody purified from the resulting culture can be used (see WO 2004/061104).

### Monoclonal Antibody Production

Monoclonal antibodies may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor, et al., Immunol. Today 4: 72 (1983)) and the EBV hybridoma technique to produce human monoclonal antibodies (see, e.g., Cole, et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote, et al., Proc. Natl. Acad. Sci. 80: 2026-2030 (1983)) or by transforming human B-cells with Epstein Barr Virus in vitro (see, e.g., Cole, et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the polypeptide. Alternatively, hybridomas expressing monoclonal antibodies can be prepared by immunizing a subject, e.g., with an isolated polypeptide comprising, or alternatively consisting essentially of, or yet further consisting of, the antigenic proteins described above, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties, binding, can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

### Phage Display Technique

As noted above, the antibodies of the present disclosure can be produced through the application of recombinant DNA and phage display technology. For example, antibodies can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse, et al., Science 246: 1275-1281, 1989) to allow rapid and effective identification of monoclonal Fab fragments with the desired specificity, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

### Alternate Methods of Antibody Production

Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFᵥs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256: 1275-1281 (1989)).

### Antibody Modifications

The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFᵥ molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

### Antibody Screening

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the the artemin receptor or artemin or any fragment or oligopeptide thereof and its specific antibody. A two-site, monoclonal-based immunoassay utilizing monoclonal antibodies specific to two non-interfering epitopes may be used, but a competitive binding assay may also be employed (Maddox et al., J. Exp. Med., 158: 1211-1216 (1983)).

### Antibody Purification

The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

### Kits

As set forth herein, the present disclosure provides kits for performing the disclosed methods as well as instructions for carrying out the methods of the present disclosure such as collecting tissue and/or performing the screen, and/or analyzing the results.

The kit comprises, or alternatively consists essentially of, or yet further consists of, an agent as describe herein (e.g., monoclonal antibodies) disclosed herein, and instructions for use. One or more of the antibodies may be labeled. The kit components, (e.g., reagents) can be packaged in a suitable container. The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### Experimental Methods

All experiments were approved by the University of Southern California Institutional Animal Care and Use Committee and performed in accordance with the recommendations of the International Association for the Study of Pain and with the National Institutes of Health (NIH) Guide for the Care and Use of Laboratory Animals. Adult (at least 8 weeks of age) wildtype or GFRα3^{-/-} mice (a gift from Dr. Brian Davis, University Of Pittsburgh, Children's Hospital of Pittsburgh, Pittsburgh, Pa.) of both sexes were used in behavioral assays. Cold, heat, and mechanical sensitivity was assayed as described (12, 70). All results from behavioral assays are shown as averages ±SEM for each group, and statistical differences were determined using the appropriate t-test for all except the evaporative cooling assay, for which statistical differences were determined using the Mann-Whitney and Wilcoxon tests.

Electronic Von Frey.

Stimulation with an electronic von Frey apparatus (IITC) was performed as previously described (22). Briefly, animals were acclimated to an elevated mesh platform for 20 min. The apparatus was fitted with a semiflexible tip and raised to the plantar surface of the hindpaw. The force at which the mouse removed the paw was measured, and average paw withdraw threshold was measured per foot from three trials per foot, alternating paws, with 3 min between trials.

Hargreaves Test.

To evaluate hindpaw sensitivity to noxious heat, the Hargreaves test was performed as previously described (23). Mice were allowed to acclimate in Plexiglas chambers placed on a glass surface heated to 32° C. (Plantar Test Apparatus, IITC) for 2 hours. A radiant heat source was focused on the hindpaw and the time to withdrawal of the paw was measured, with an interstimulation period of 5 min per mouse, alternating paws, for a total of four trials per paw for each time point. The readings from the four trials were averaged to give the withdrawal latency.

Cold Plantar Assay.

In order to assess cold sensitivity in a manner comparable to the Hargreaves test, the cold plantar assay was performed (70). Mice were acclimated in Plexiglas chambers placed on a glass plate for 3 hours. A dry ice pellet was then applied to the hindpaw of the mouse through the glass, and the latency to withdrawal was recorded, with an interstimulation period of 5 min per mouse, alternating paws, for a total of four trials per paw for each time point. The thickness of the glass (6 mm) was such that latencies were 8-12 s at baseline.

Evaporative Cooling Assay.

To evaluate cold sensitivity of the hindpaws, the acetone evaporative cooling assay was performed as previously described (22). Briefly, mice were acclimated for 20 min in an elevated chamber with a mesh floor (IITC), and a small drop of acetone (˜50 μl) was deposited on the paw plantar surface of the hindpaw using the tip of a 10 cc syringe. Mice were tested with an interstimulation period of 4 min per mouse, alternating paws between stimulations, for a total of four trials per paw for each time point. Responses were video recorded for later quantification by an observer blind to genotype and the solution injected. Behaviors were scored according to the magnitude of the response along the following scale: O-no response; 1-brief lift, sniff, flick, or startle; 2-jumping, paw shaking; 3-multiple lifts, paw lick; 4-prolonged paw lifting, licking, shaking, or jumping; 5-paw guarding (12, 14, 22).

Artemin and NGF Injections.

Artemin and NGF injections to the hindpaw were performed as described previously (23). Briefly, mice were lightly anesthetized with isofluorane, and 20 μl of vehicle (0.9% saline), artemin (R&D Systems, diluted to 10m/ml in saline), or NGF (Life Technologies, diluted to 10m/ml in saline) were administered by intraplantar injection.

Pain Models.

Inflammation was induced unilaterally by intraplantar injection of 20 μl of complete Freund's adjuvant (CFA) into the hindpaw. Neuropathic pain was induced using chronic constriction injury (CCI) model or by the chemotherapeutic (12, 38). Mice were tested 2 days after injection for cold and mechanical sensitivity, and 3 days after injection for heat sensitivity.

The chronic constriction injury (CCI) model was used to evaluate neuropathic pain as previously described (12, 38). Mice were anesthetized with 5% isofluorane for induction and 3% isofluorane for maintenance. The surface of the left hindlimb was shaved and sterilized. A 2 cm incision was made in the skin, and the muscles overlaying the sciatic nerve were gently retracted. Three loose ligatures of 6-0 chromic gut were tied around the nerve proximal to the trifurcation site. The muscles were replaced, and the wound was closed with VetBond tissue adhesive (3M) and swabbed with topical antibiotic. Animals were monitored for infection and proper wound healing and were tested on day 7 post-surgery.

The oxaliplatin model was used to test chemotherapeutic-induced neuropathic pain. Oxaliplatin (Sigma-Aldrich, dissolved in 5% glucose in saline) was administered by intraperitoneal injection at a concentration of 3 mg/kg. Mice were tested on day 3 post-injection for von Frey, day 4 for evaporative cooling, and day 7 for cold plantar assays.

Antibody Administration.

Anti-artemin antibody (R&D Systems, MAB1085, Rat IgG2A) or isotype control (MAB006, Rat IgG2A) was dissolved in saline and injected subcutaneously to the scruff at a concentration of 10 mg/kg. For CFA, antibodies were injected on day 2 post-CFA for acetone, cold plantar and von Frey assays, and day 3 for Hargreaves. For oxaliplatin, antibodies were injected on day 7 post-oxaliplatin for cold plantar and von Frey. In all cases, responses were measured 4h post-antibody injection. For oxaliplatin, an additional time point for the cold plantar assay was assessed 24h after antibody injection. For NGF injections, mice were injected with the anti-artemin antibody, then unilateral plantar NGF injections were performed as described above 1 hr later.

In order to study the neurochemical profile of TRPM8 neurons in GFRα3^{-/-} sensory ganglia, TRPM8^{GFP} mice were crossed with GFRα3^{-/-} line to obtain TRPM8^{GFP}-GFRα3^{+/-} mice, which were then crossed with each other. As has been previously described (9, 23), immunolabeling was carried out on sections of L4-L6 dorsal root ganglia (DRG) dissected from adult (at least 8 weeks old) TRPM8^{GFP} or TRPM8^{GFP}-GFRα3^{-/-} mice. Primary antibodies were diluted in a working solution (0.3% Triton X-100 and 1-5% normal donkey serum in PBS, pH 7.4). Primary antibodies and dilutions used are as follows: 1:500 chicken anti-GFP (GFP-1020; Ayes Labs, Tigard, Oreg.), 1:200 goat anti-GFRα3 (GT15123; Neuromics, Edina, Minn.), 1:500 guinea pig anti-CGRP (T-5027; Bachem, Torrance, Calif.), 1:500 rabbit anti-peripherin (AB1530; Millipore, Temecula, Calif.), 1:500 rabbit anti-NF200 (N4142; Sigma, St. Louis, Mo.), 1:500 guinea pig anti-TRPV1 (a gift from D. Julius, University of California San Francisco, San Francisco, Calif.). Sections were incubated with primary antibody solutions at 4° C. for 24-48 hrs, then washed and incubated with the appropriate Alexa Fluor-conjugated secondary antibody solutions (1:1000 Alexa Fluor 350, Alexa Fluor 488 or Alexa Fluor 594, Molecular Probes) at room temperature for 1-2 hours. To detect D34 binding, 1:2000 Griffonia simplicifolia isolectin GS-D34-Alexa 568 (1-21412; Life Technologies, Carlsbad, Calif.) was added to the secondary antibody solutions. Slides were washed and mounted in ProLong Gold reagent (Life Technologies). Digital images were acquired using a Zeiss Axio Imager.M2 with Apotome attachment and Axiovision software, and analyzed using ImageJ. Quantification is reported as percent overlap between two or three markers, plus or minus the standard error between fields (71), and statistical differences were determined using the unpaired t-test.

qPCR analysis

To determine whether the lack of GFRα3^{-/-} receptors influenced the expression levels of genes implicated in nociception and thermal sensitivity, quantitative PCR analysis was performed. Dorsal root ganglia were harvested from Gfrα3^{-/-} mice or wildtype littermate controls and placed in RNAlater buffer (Qiagen), and total RNA was purified using the RNeasy Mini kit (Qiagen) with in-column DNA digestion according to the manufacturer's instructions. The iScript cDNA synthesis kit (Bio-Rad) was used to synthesize cDNA from the purified RNA samples, and qPCR was performed using Ssofast EvaGreen supermix (Bio-Rad) and a Bio-Rad CFX96 detection system. All samples were run in duplicate, and ΔCt values normalized to the reference standard were obtained as follows: ΔCt=Ct (gene of interest)−Ct (GAPDH).

Relative changes in expression were calculated using the ΔΔCt method. The primers used are listed below:

| | | |
|---|---|---|
| TRPM8 | FWD: 5′ GCTGCCTGAAGAGGAAATTG 3′ | (600 bp) |
| | REV: 5′ GCCCAGATGAAGAGAGCTTG 3′ | |
| TRPV1 | FWD: 5′ CTTCAGCCATCGCAAGGAGT 3′ | (769 bp) |
| | REV: 5′ GTTTCTCCCTGAAACTCGGC 3′ | |
| TRPA1 | FWD: 5′ ACAAGAAGTACCAAACATTGACACA 3′ | (243 bp) |
| | REV: 5′ TTAACTGCGTTTAAGACAAAATTCC 3′ | |
| Nav | FWD: 5′ TCCCAGGCCTGAAGACAATC 3′ | (445 bp) |
| 1.6 | REV: 5′ GCTCGTAAGGTCAGCTGGTAT 3′ | |
| Nav | FWD: 5′ ACCAGCAGACACTCCGGGCT 3′ | (457 bp) |
| 1.8 | REV: 5′ ACGTCTTGGCTGGGTGCTCG 3′ | |
| TREK1 | FWD: 5′ GAGATACAGACTGCTGGCATAG 3′ | (229 bp) |
| | REV: 5′ GTAGATGTAAGTACGGGCACAG 3′ | |
| TRAAK | FWD: 5′ TTATGTACCCGGCGATGGCACCGG 3′ | (127 bp) |
| | REV: 5′ TGCTCGCAACCAGTTGCCGATG 3′ | |
| TASK3 | FWD: 5′ GGAACACCTACTTCCGGTCC 3′ | (169 bp) |
| | REV: 5′ GGGAAAGAGGAGTTGGGACAAT 3′ | |
| GAPDH | FWD: 5′ TGTAGACCATGTAGTGAGGTCA 3′ | (123 bp) |
| a | REV: 5′ AGGTCGGTGTGAACGGATTTG 3′ | |

Previously, it was shown that intraplantar hindpaw injections of artemin and NGF induces a robust and transient TRPM8-dependent cold allodynia (23). The NGF/TrkA signaling pathways and its requirement in sensory neuron development and sensitization are well established (1), but how GFRαreceptors induce sensory neuron sensitization is poorly understood. Therefore, to determine how artemin leads to cold pain, Applicant first examined acute sensitivity of mice lacking the artemin receptor GFRα3 (Gfrα3^{-/-}) to thermal or mechanical stimuli which, to the best of Applicant's knowledge, has not been reported for these animals (34, 35).

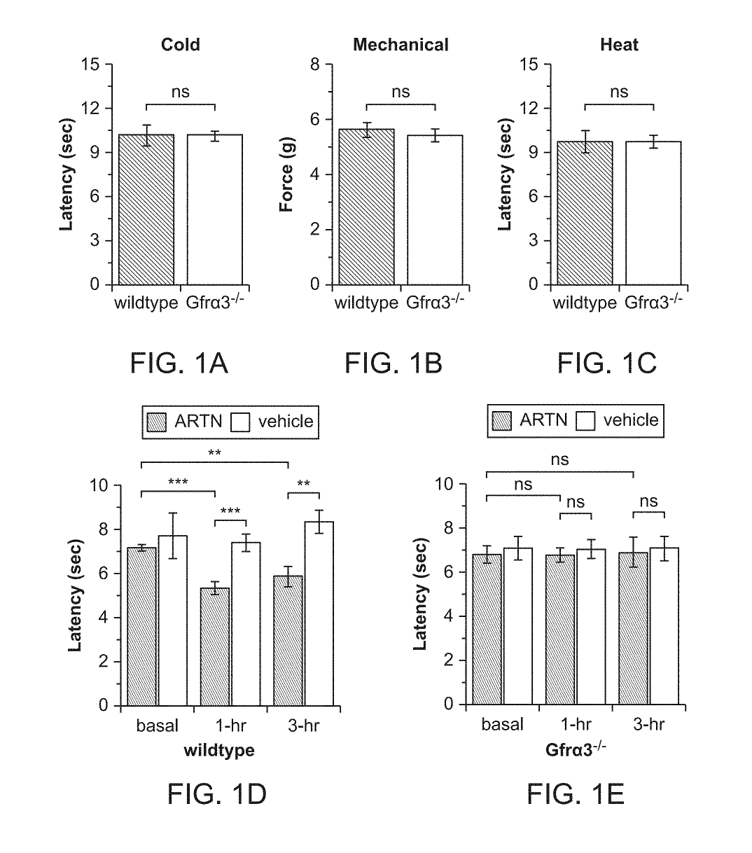

Using the cold plantar, von Frey (mechanical), and Hargreaves (radiant heat) assays Applicant compared thermal and mechanically evoked behaviors of wildtype and Gfrα3^{-/-} mouse littermates, finding no differences between the two genotypes (FIGS. 1A-1C; p>0.05). Among the four distinct GFL a receptor subtypes (GFRα), artemin has been reported to be highly selective for GFRα3 (36), but has also been suggested to cross-react with other GFL receptors (37).

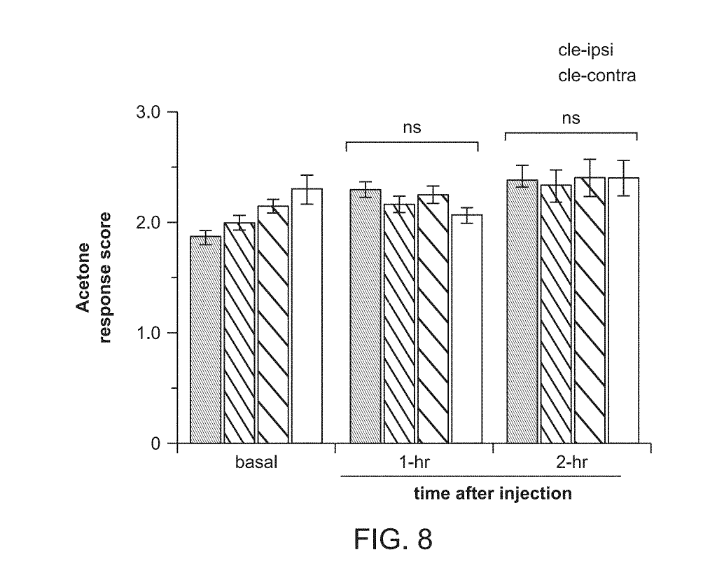

Therefore to determine if artemin's effects on cold sensitivity are GFRα3-specific, Applicant examined cold sensitivity after intraplantar artemin injections in both wildtype and Gfrα3^{-/-} mice. In wildtypes, the latency to a paw withdrawal from a radiant cold stimulus using the cold plantar assay was significantly decreased at 1- and 3-hrs after artemin injection (FIG. 1D, p<0.001 at 1 hr versus basal or vehicle-injected; p<0.01 at 3 hr). However, consistent with this ligand's selectivity for GFRα3 (36), hindpaw injections of artemin failed to alter cold sensitivity in Gfrα3^{-/-} mice (FIG. 1E, p>0.05). Similar results were observed in Gfrα3^{-/-} mice using the evaporative cooling assay (FIG. 8). Taken together, these data show that acute nociceptive behaviors are not altered in GFRα3-deficient mice and that artemin-induced cold hypersensitivity is GFRα3-dependent.

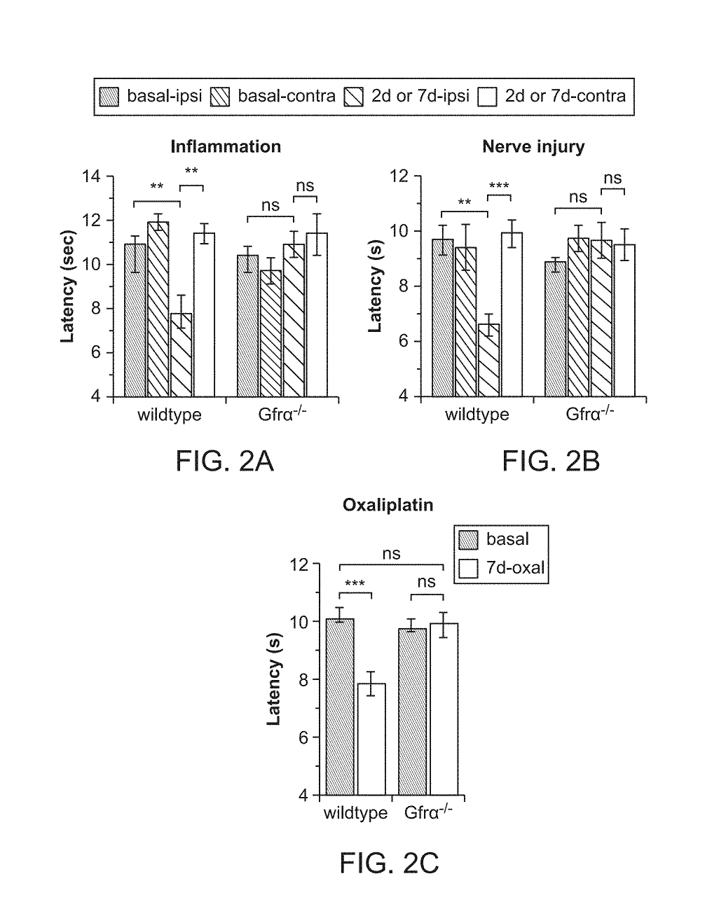

Next, Applicant set out to determine the role of GFRα3 in pathological cold pain. To test this, Applicant examined cold sensitivity in adult wildtype and Gfrα3^{-/-} littermates in classical models of inflammation, nerve injury, and chemotherapeutic neuropathic pain (12, 22). Wildtype mice show robust cold allodynia 2 days after unilateral injections of the inflammatory agent Complete Freund's Adjuvant (CFA) (FIG. 2a; p<0.01 pre- vs. post-CFA or ipsilateral vs. contralateral), as Applicant and others have previously reported (12, 14, 22). In contrast, Gfrα3^{-/-} mice show no differences in their hindpaw lift latencies between the ipsilateral (inflamed) and the contralateral (control) sides, nor were there differences in their sensitivity compared to the basal, pre-inflamed state (FIG. 2A; p>0.05). To determine the general nature of this inability of Gfrα3^{-/-} mice to mount a cold allodynic response after injury, Applicant also examined animals with neuropathic pain caused by chronic constriction of the sciatic nerve (CCI) (38). As with inflammation, cold allodynia was observed in wildtype animals (FIG. 2B; p<0.01 pre- vs. 7d-post injury; p<0.001 ipsi vs. contra), but cold sensitivity was remarkably unchanged in Gfrα3^{-/-} mice (ipsi vs. contra, pre- vs. 7d-post injury, p>0.05). Lastly, one of the major side effects of platin-based chemotherapeutics is cold pain (39), a phenotype that can be modeled in mice given a single systemic injection of oxaliplatin (12, 22). As with the previous pain models, the cold allodynia observed in wildtype mice (p<0.001 basal vs. 7d-post injection) was completely absent in mice null for GFRα3 (FIG. 2C; p>0.05 pre- vs. post-injection and Gfrα3^{-/-} post-injection vs. wildtype mice pre-injection). Applicant observed similar results in all three pathological pain models when cold sensitivity was determined by evaporative cooling (FIGS. 2A-C).

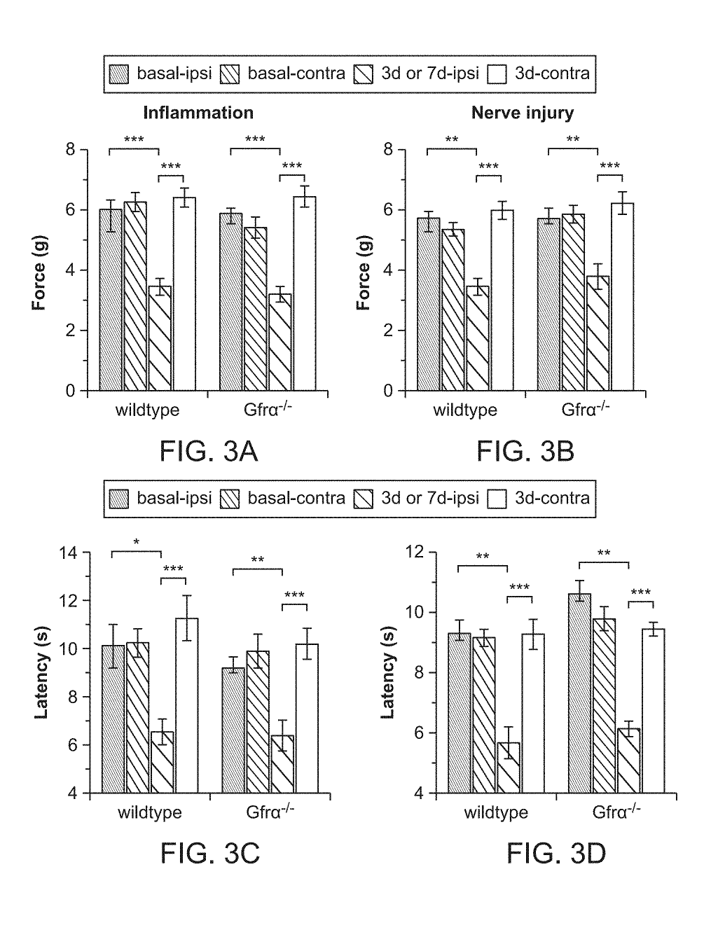

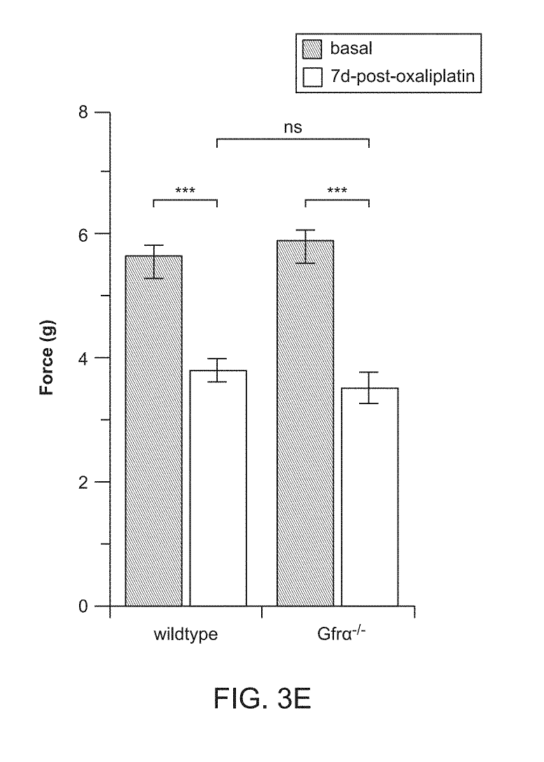

Next, Applicant asked how specific is the role of GFRα3 signaling for cold pain versus other pain modalities? To address this question Applicant examined mechanical and radiant heat evoked responses in wildtype and Gfrα3^{-/-} mice in the three pain models tested previously. In striking contrast to cold-evoked behaviors, Applicant observed robust mechanical hyperalgesia in both wildtype and Gfrα3^{-/-} mice in the context of inflammation (FIG. 3A; p<0.001 pre- vs. post-CFA or ipsi vs. contra), with nerve injury (FIG. 3B; p<0.01 pre- vs. post-injury; p<0.001 ipsi vs. contra), or with oxaliplatin-induced polyneuropathy (FIG. 3C; p<0.001 basal vs. post-injection). Next, Applicant examined heat hyperalgesia in both the CFA inflammatory and CCI neuropathic pain models (oxaliplatin does not induce heat hyperalgesia). As with mechanical pain, Applicant observed strong heat hyperalgesia in both wildtype and Gfrα3^{-/-} mice with inflammation (FIG. 3D; p<0.05 and p<0.01 pre- vs. post-CFA for wildtype and Gfrα3^{-/-} mice, respectively; p<0.001 ipsi vs. contra) or with irritation of the sciatic nerve (FIG. 3E; p<0.01 pre- vs. post-injury; p<0.001 ipsi vs. contra). Furthermore, there was no difference between the levels of mechanical and heat hyperalgesia between the two genotypes (p>0.05), demonstrating that GFRα3 is not absolutely required for heat and mechanical pain as it is for cold. These remarkable results demonstrate that, unlike the redundant nature of heat or mechanical sensitization, which are mediated by several algogenic receptors (1), injury-evoked cold allodynia, both inflammatory and neuropathic, requires the artemin receptor GFRα3.

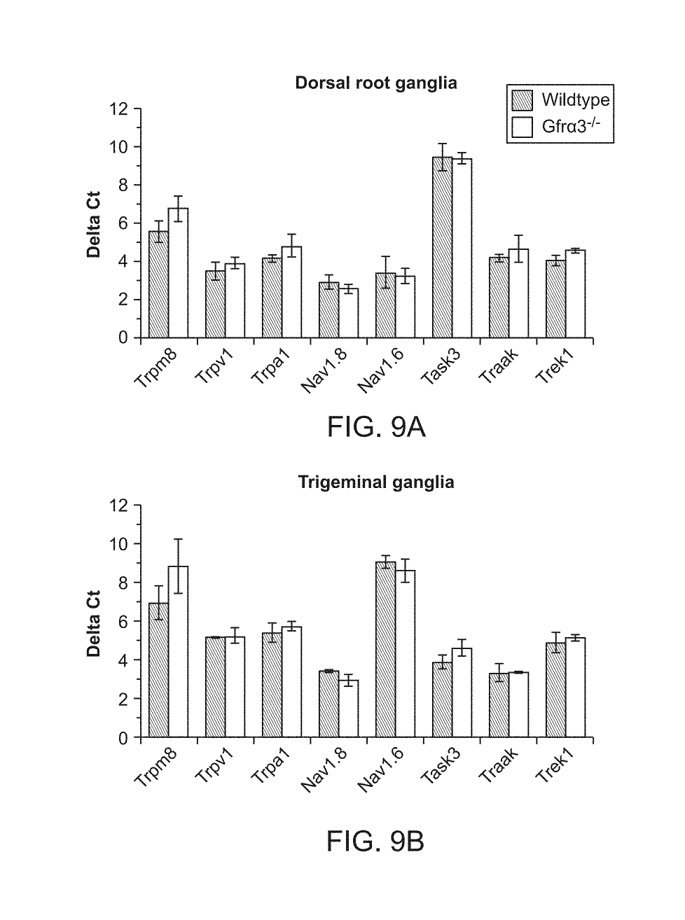

Experimentally-induced over-expression of artemin in peripheral tissues has been shown to lead to heat and mechanical hyperalgesia, as well as altered expression of molecules involved in sensory transduction (32, 40). However, these studies involved either genetically-induced artemin over-expression in the periphery (40), or multiple plantar injections of exogenous artemin (32), making it unclear if artemin/GFRα3 signaling influences afferent expression phenotypes under physiological conditions. Thus, to determine if the lack of a cold allodynic phenotype in Gfrα3^{-/-} mice is a result of alterations in sensory afferent development due to the absence of GFRα3, Applicant used quantitative PCR to determine expression of an array markers involved in cold thermosensation (17). In adult DRG (FIG. 9A) and trigeminal neurons (FIG. 9B), Applicant observed no differences (p>0.05) in transcript expression of either known thermosensory receptors (Trpm8, Trpa1, or Trpv1) or channels implicated in excitability of thermosensory afferents (Nav1.8, 1.6, Task3, Traak, and Trek1) in wildtype and Gfrα3^{-/-} mice (41).

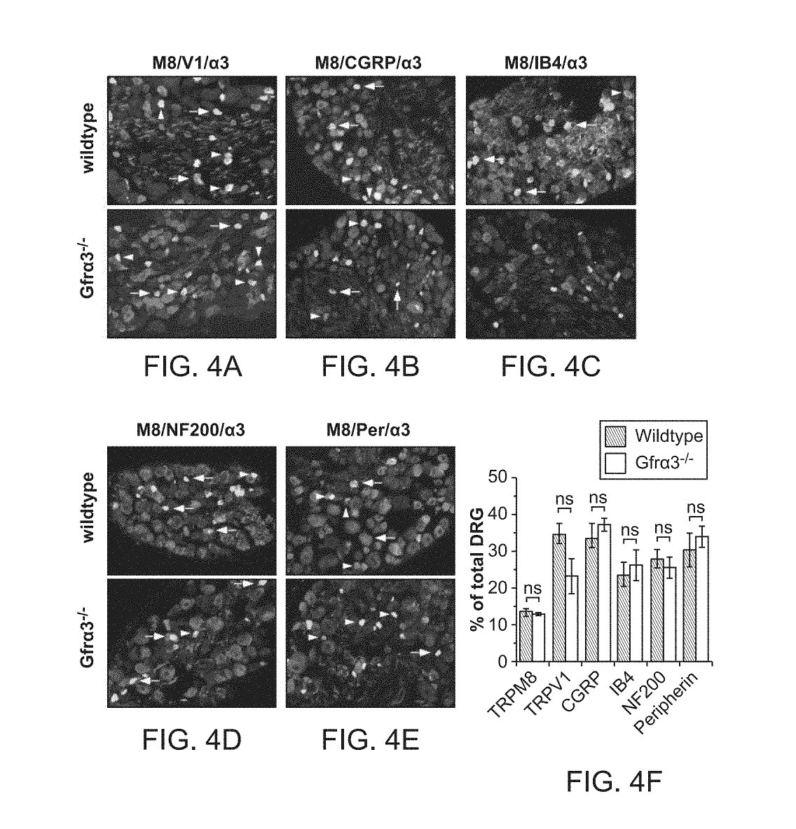

Next, using immunohistochemistry Applicant examined the protein expression phenotype of Gfrα3^{-/-} mice, first establishing that immunoreactivity for GFRα3 was absent in adult L4-L6 DRG from these animals (FIG. 4). TRPM8 is the principle cold thermoreceptor in mammals and Applicant has shown that it is required for artemin-mediated cold allodynia (8, 23). GFRα3 is expressed in one-half of TRPM8⁺ DRG neurons (23), but consistent with Applicant's transcript expression analysis, Applicant observed no difference in the number of TRPM8− positive neurons between wildtype and Gfrα3^{-/-} mice (FIG. 4F, p>0.05). GFRα3 is found exclusively in TRPV1-positive afferents (40, 42), but Applicant observed no difference in TRPV1 expression in Gfrα3^{-/-} mice (FIGS. 4A, 4F). Moreover, the number of neurons immunoreactive to antibodies to calcitonin gene-related peptide (CGRP), a marker of peptidergic nociceptors (FIG. 4B, 4F), and bound the non-peptidergic neuronal marker isolectin IB4 (FIG. 4C, 4F) were similar. There was also no difference in fiber-type distribution as the number of neurons labeled for the A-fiber marker NF200 (FIG. 4D, 4F) and the C-fiber marker peripherin (FIG. 4E, 4F) were similar between genotypes. These results demonstrate that the development of DRG neurons is not influenced by GFRα3 signaling, results consistent with prior analyses of these mice, as well as those lacking artemin expression (34, 35).

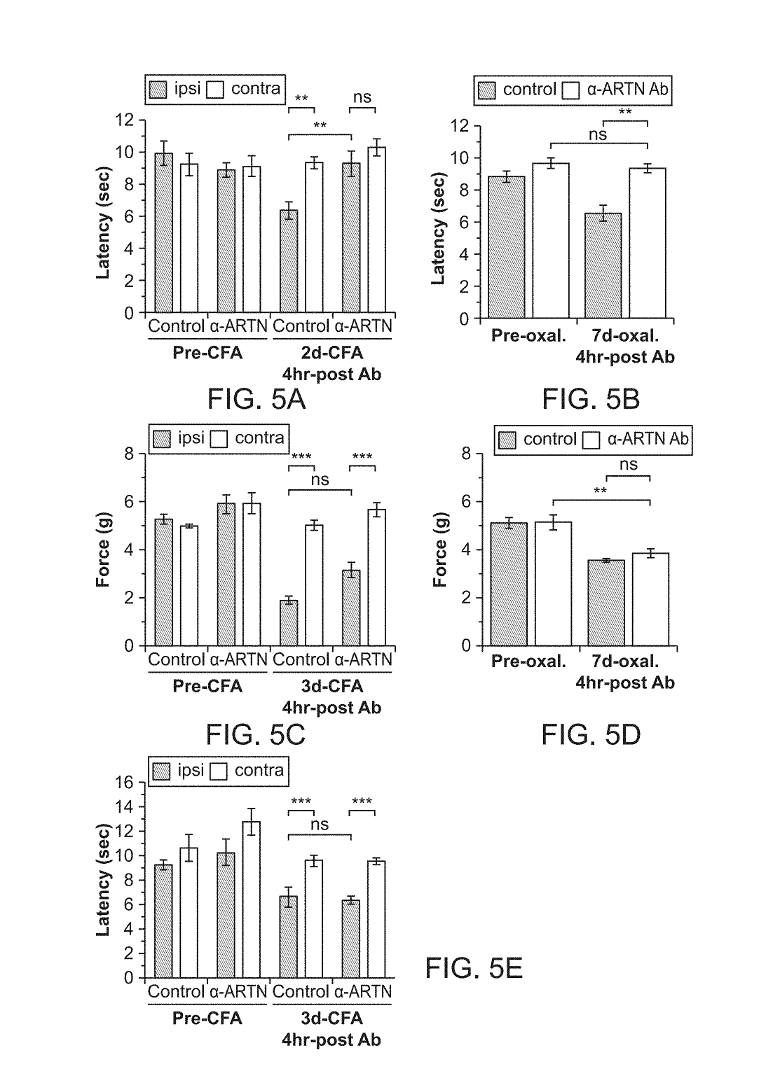

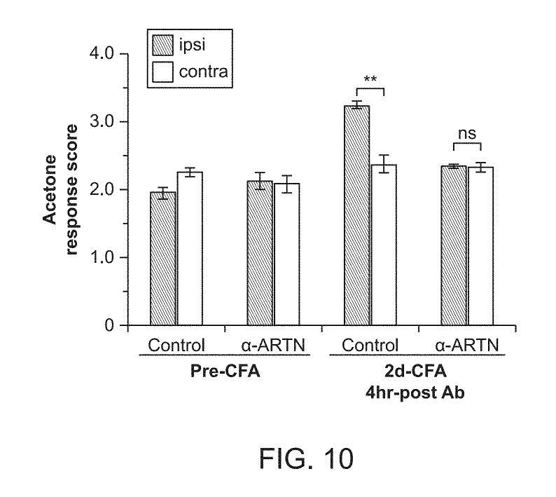

Applicant's results suggest that cold allodynia is specifically mediated by artemin signaling via its receptor GFRα3 which likely functions upstream of molecules involved in cold transduction, highlighting what appears to be a highly specific pathway leading to cold pain. Due to this extraordinary specificity, Applicant hypothesized that in vivo neutralizing artemin in mouse models of inflammatory and neuropathic pain could selectively block cold allodynia, even that which is localized at or near a site of injury, whereas heat and mechanical pain would remain intact. Artemin neutralizing antibodies are known to effectively inhibit binding of artemin with GFRα3 in vivo and serve as a potential pharmacological mechanism to ameliorate or prevent the effects of artemin exposure (31, 43, 44). Therefore, Applicant tested whether a systemic injection of an established artemin-neutralizing monoclonal antibody could reverse inflammatory and neuropathic pain. Remarkably, both inflammatory (FIG. 5A) and oxaliplatin-induced (FIG. 5B) cold allodynia was ameliorated in wildtype mice 4 hrs after intradermal injection (10 mg/kg) of the anti-artemin antibody MAB1085 (p>0.05, ipsi vs. contra), whereas mice injected with an isotype control antibody remained sensitized to cold (FIGS. 5A, 5B; p<0.01, ipsi vs. contra or pre- vs. post-injection). Applicant observed a similar reduction in cold allodynia in mice tested with the evaporative cooling assay (FIG. 10). The block of cold pain was transient as sensitization was evident at 24 hr post-MAB1085 treatment (not shown), consistent with prior bioactivity analyses of artemin-neutralizing antibodies administered in this fasion (31).

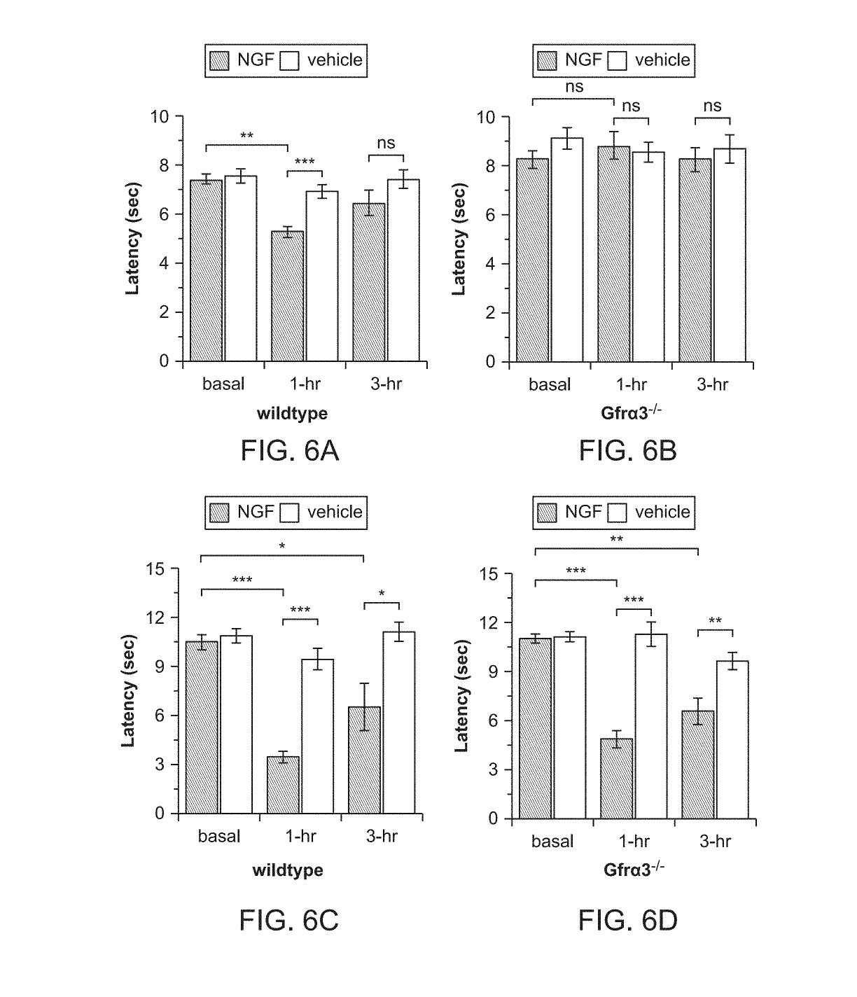

Moreover, and in agreement with Applicant's genetic analysis, treatment with MAB1085 had no effect on mechanical (FIGS. 6C, 6D; p<0.001) or heat (FIG. 6E; p<0.001) hyperalgesia observed in the ipsilateral versus contralateral hindpaws, nor was the level of hyperalgesia different between control and MAB1085 treated mice (p>0.05). These data demonstrate that artemin-neutralizing antibodies can reverse multiple types of injury-induced cold pain in an effective and highly specific manner.

To date, unlike heat and mechanical hyperalgesia, only artemin, and to a lesser extent NGF, have been found to induce cold hypersensitivity in mice when administered via intraplantar injections (18, 19, 23). NGF signals via the tyrosine kinase TrkA and is a major mediator of heat hyperalgesia through sensitization of the heat-gated capsaicin receptor TRPV1 (45, 46). Based on the extensive literature on NGF/TrkA signaling Applicant expected that NGF-induced cold sensitization was mediated via TrkA through cellular signal transduction cascades that sensitize molecules involved in cold transduction. However, to Applicant's surprise, Applicant found that cold allodynia observed in wildtype mice 1 hr after intraplantar NGF-injection (FIG. 6A, p<0.01 at 1 hr versus basal and vehicle-injected) was absent in Gfrα3^{-/-} mice injected with NGF (FIG. 6B, p>0.05 at 1- and 3 hr post injection; p>0.05 NGF vs. veh. at all times tested), results similar to that observed after artemin injection. To ensure that this absence of cold allodynia was not due to a general reduction in NGF-sensitization in these mice Applicant tested heat hyperalgesia, finding that, consistent with Applicant's analyses of inflammatory and neuropathic pain, heat hyperalgesia remained intact in Gfrα3^{-/-} mice (FIG. 6D., p<0.001 at 1 hr post injection and NGF vs. veh.; p<0.01 at 3 hrs post-inj and NGF vs. veh.) and similar to that observed in wildtype animals (FIG. 6C). Thus, these results demonstrate that NGF-induced cold allodynia requires GFRα3, suggesting for the first time that cellular mechanisms leading to cold hypersensitivity converge on GFRα3.

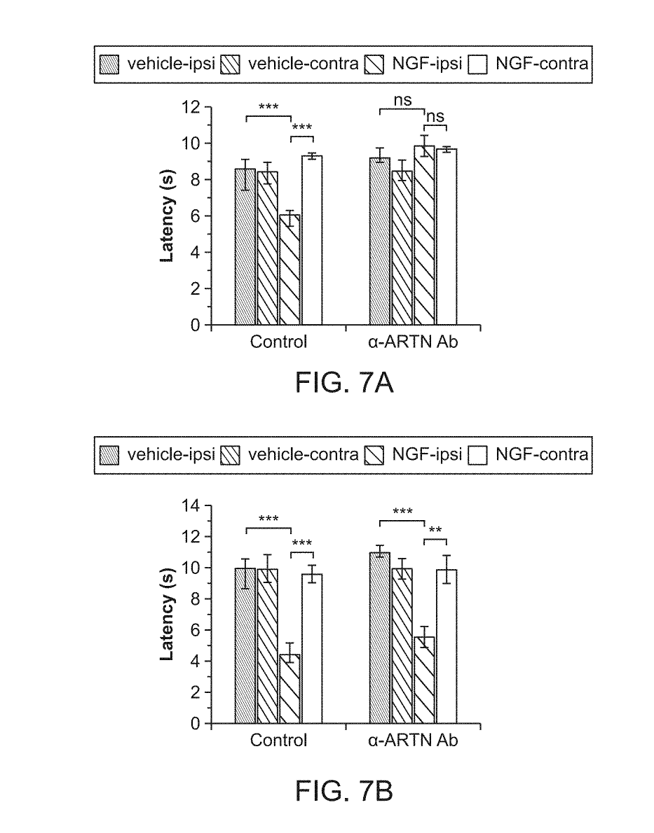

How then does NGF prompt GFRα3-dependent cold allodynia? In addition to direct sensitization of nociceptors, NGF also acts indirectly thru activation of various peripheral cell types and the subsequent release a host of inflammatory mediators which, in turn, sensitize sensory afferents (25, 47-49). Several inflammatory conditions stimulate artemin release from a number of peripheral cell types, including keratinocytes, fibroblasts, and immune cells (32, 50, 51), and Applicant hypothesized that NGF-induced cold allodynia was mediated by NGF indirectly promoting artemin release. To test this, Applicant again used artemin-neutralizing antibodies and found that NGF-evoked cold allodynia observed in control mice (FIG. 7A, p<0.001 NGF vs. veh, ipsi vs. contra), measured 1 hr after intraplantar NGF injections, was blocked when MAB1085 was administered systemically 1 hr prior to NGF treatment (FIG. 7A, p>0.05 NGF vs. veh, ipsi vs. contra). This absence of NGF-evoked sensitization was again modality specific as MAB1085 had no effect on heat hyperalgesia when compared to controls (FIG. 7B, p<0.001 NGF vs. veh, ipsi vs. contra for both conditions). Thus, these results show that NGF-induced cold allodynia is mediated by artemin signaling through its cognate cellular receptor GFRα3, further validating the necessity and specificity of this signaling pathway on pathological cold pain.

To Applicant's knowledge, this is the first rigorous report of nociception in mice lacking GFRα3, and Applicant's results are consistent with prior studies that found no discernable phenotype in peripheral sensory ganglia in both artemin and GFRα3-null mice (34, 35). The lack of any salient somatosensory abnormalities in naïve Gfrα3^{-/-} mice suggests that the receptor has no substantial role in sensory nervous system development, despite the fact that it is expressed in ˜20% of adult DRG neurons.

Nonetheless, Applicant now demonstrates the necessity of GFRα3 in pathological cold pain induced by an important inflammatory mediator (NGF), by inflammation itself, and in two distinct forms of neuropathic pain. What is a remarkable and seminal result of Applicant's study is that injury-induced cold allodynia of multiple etiologies is totally dependent on GFRα3, unlike the redundant nature of heat and mechanical pain that signal through a diverse repertoire of cell surface receptors (1). NGF, GDNF, artemin, protons, bradykinin and histamine, to name a few, are all capable of potentiating TRPV1 responses (7, 17, 30, 52) and are responsible for the development of heat hyperalgesia. However, of these proalgesics, only artemin and NGF were found to induce cold allodynia in a manner similar to that found after injury (23). Here Applicant shows that both proalgesics promote cold pain via GFRα3, and that NGF-induced cold allodynia occurs through a mechanism that involves artemin since it is ameliorated with artemin neutralization. The latter results are consistent with an indirect action of NGF on nociceptors in which NGF, in addition to binding to neuronal TrkA receptors, also activates immune cells to release a host of inflammatory mediators (25, 47).

The signal transduction mechanisms that lead to cold sensitization after artemin activation of GFRα3 remains unclear. Applicant recently reported that artemin and NGF-evoked cold allodynia was dependent on TRPM8 channels, but a direct action of these inflammatory mediators on TRPM8 channel activity has yet to be reported. The molecular processes whereby NGF/TrkA activation lead to sensitization of TRPV1 channels and heat hyperalgesia are well documented (7) but, to date, the molecular nature of GFL signaling on nociception has yet to be elucidated. For example, as Applicant and others have shown acute exposure of GFLs, including artemin, in vivo leads to heat hyperalgesia (23, 30, 50). Similarly, GFLs potentiate capsaicin responses in dissociated DRG neurons recorded by Ca²⁺ imaging, demonstrating sensitization of TRPV1⁺ cells (30). However, specific changes in TRPV1 channel activity have not been reported and the preponderance of data suggests that artemin-induced heat hyperalgesia is due to increased TRPV1 expression (32, 40, 53, 54). Moreover, unlike the established TRPM8-dependence of artemin-evoked cold allodynia (23), or the necessity of TRPV1 for NGF-induced heat hyperalgesia (45), the molecule determinants of GFL-evoked alterations in nociception in vivo are unknown (55).

What then underlies artemin and GFRα3-dependent cold pain? As an extraceullular receptor, GFRα3 must bind to a transmembrane protein in order to transduce a signal, which in many systems is the tyrosine kinase Ret (56). However, recent evidence has uncovered GFRαactions that are Ret-independent, and while the exact transduction mechanisms underlying the GFL signaling in the absence of Ret have yet to be elucidated, both the neural cell adhesion molecules (NCAM) and integrin β1 are reported to act as co-receptors with GFRαs (57, 58). Nonetheless, these receptor complexes signal intracellularly through similar molecular mechanisms, including protein kinases (MAPK, p38/JNK, PI3K, src family, PKA, PKC) and phospholipases (PLCβ, PLCγ) (28, 56, 57), pathways also known to modulate heat sensitivity and TRPV1 function (59-63). Artemin- and NGF-induced sensitization of cold responses is TRPM8-dependent and multiple studies have shown that TRPM8 plays a role in the development of cold allodynia (12, 14, 22, 23, 64). However, inflammatory cold allodynia is also diminished by both TRPA1 antagonist and reduced TRPA1 transcript expression, and cold hypersensitivity can be induced by TRPA1 agonism in wild-type but not Trpa1^{-/-} mice (20, 21). Thus, both TRPM8 and TRPA1 are involved in pathological cold pain, thought it should be noted that artemin directly inhibit TRPA1 channels (33), but it is unknown how channel inhibition influences any potential changes to TRPA1 function downstream of GFRα3 activation.

Lastly, ion channels involved in neuronal excitability have been implicated in cold pain. For example, two-pore, non-gated potassium channels contribute to cold pain as they are down-regulated after oxaliplatin treatment, thereby leading to enhanced neuronal excitability (65, 66). Moreover, antagonism of the voltage-gated sodium channel Nav1.6 attenuated neuropathic cold allodynia (67). Thus, the mechanisms that potentiate cold responses at the molecular and cellular level are diverse, but Applicant's data strongly suggest that future studies into cold pain should center around the effect of GFRα3 activation on these pathways.

Finally, Applicant provides evidence here that artemin and GFRα3 are potential therapeutic targets for conditions in which cold alloydnia is a symptom. Artemin-neutralizing antibodies can ameliorate both inflammatory and neuropathic cold pain that has already been established in the animal, suggesting that interfering with artemin is a potential therapeutic strategy for this pain modality. Applicant's results are consistent with other recent reports which suggest that artemin neutralization may work beyond cold pain as anti-artemin antibodies were found to inhibit non-inflammatory heat hyperalgesia in the tongue and reduce bladder hyperalgesia (31, 43, 44). Moreover, this approach is analogous to therapeutic interventions to block pain with NGF-neutralizing antibodies that are currently ongoing (68, 69). The finding that anti-artemin antibodies can block oxaliplatin-induced cold allodynia provides a particularly promising prospect for clinical application. When taken as a whole, these studies show that, unlike the broad range of mediators of mechanical and heat pain, the exacerbation of cold pain after injury is mediated exclusively by the GFL artemin and its receptor GFRα3, providing the first evidence of a proalgesic agent singularly required for cold sensitization. Thus, artemin and GFRα3 can be considered as valuable therapeutic targets due to their effectiveness and specificity to cold pain.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Basbaum A I, Bautista D M, Scherrer G, & Julius D (2009) Cellular and molecular mechanisms of pain. Cell 139(2):267-284.
2. Knowlton W M & McKemy D D (2011) TRPM8: from cold to cancer, peppermint to pain. Curr Pharm Biotechnol 12(1):68-77.
3. Svendsen K B, Jensen T S, Hansen H J, & Bach F W (2005) Sensory function and quality of life in patients with multiple sclerosis and pain. Pain 114(3):473-481.
4. Greenspan J D, Ohara S, Sarlani E, & Lenz F A (2004) Allodynia in patients with post-stroke central pain (CPSP) studied by statistical quantitative sensory testing within individuals. Pain 109(3):357-366.
5. Lampert A, O'Reilly A O, Reeh P, & Leffler A (2010) Sodium channelopathies and pain. Pflugers Arch 460(2):249-263.
6. Ji R R, Xu Z Z, & Gao Y J (2014) Emerging targets in neuroinflammation-driven chronic pain. Nature reviews. Drug discovery 13(7):533-548.
7. Julius D (2013) TRP channels and pain. Annu Rev Cell Dev Biol 29:355-384.
8. McKemy D D, Neuhausser W M, & Julius D (2002) Identification of a cold receptor reveals a general role for TRP channels in thermosensation. Nature 416(6876):52-58.
9. Takashima Y, et al. (2007) Diversity in the neural circuitry of cold sensing revealed by genetic axonal labeling of transient receptor potential melastatin 8 neurons. The Journal of neuroscience: the official journal of the Society for Neuroscience 27(51):14147-14157.
10. Proudfoot C J, et al. (2006) Analgesia Mediated by the TRPM8 Cold Receptor in Chronic Neuropathic Pain. Curr Biol 16(16):1591-1605.
11. Knowlton W M, Fisher A, Bautista D M, & McKemy D D (2010) TRPM8, but not TRPA1, is required for neural and behavioral responses to acute noxious cold temperatures and cold-mimetics in vivo. Pain 150(2):340-350.
12. Knowlton W M, et al. (2013) A Sensory-Labeled Line for Cold: TRPM8-Expressing Sensory Neurons Define the Cellular Basis for Cold, Cold Pain, and Cooling-Mediated Analgesia. The Journal of neuroscience: the official journal of the Society for Neuroscience 33(7):2837-2848.
13. Bautista D M, et al. (2007) The menthol receptor TRPM8 is the principal detector of environmental cold. Nature 448(7150):204-208.
14. Colburn R W, et al. (2007) Attenuated cold sensitivity in TRPM8 null mice. Neuron 54(3):379-386.
15. Dhaka A, et al. (2007) TRPM8 is required for cold sensation in mice. Neuron 54(3):371-378.
16. Caterina M J, et al. (2000) Impaired nociception and pain sensation in mice lacking the capsaicin receptor. Science 288(5464):306-313.
17. Palkar R, Lippoldt E K, & McKemy D D (2015) The molecular and cellular basis of thermosensation in mammals. Current opinion in neurobiology 34C:14-19.
18. Zhang X, et al. (2012) Direct inhibition of the cold-activated TRPM8 ion channel by Galphaq. Nat Cell Biol 14(8):851-858.
19. Linte R M, Ciobanu C, Reid G, & Babes A (2007) Desensitization of cold- and menthol-sensitive rat dorsal root ganglion neurones by inflammatory mediators. Exp Brain Res 178(1):89-98.
20. da Costa D S, et al. (2010) The involvement of the transient receptor potential A1 (TRPA1) in the maintenance of mechanical and cold hyperalgesia in persistent inflammation. Pain 148(3):431-437.
21. del Camino D, et al. (2010) TRPA1 contributes to cold hypersensitivity. The Journal of neuroscience: the official journal of the Society for Neuroscience 30(45):15165-15174.
22. Knowlton W M, Daniels R L, Palkar R, McCoy D D, & McKemy D D (2011) Pharmacological Blockade of TRPM8 Ion Channels Alters Cold and Cold Pain Responses in Mice. PLoS One 6(9):e25894.
23. Lippoldt E K, Elmes R R, McCoy D D, Knowlton W M, & McKemy D D (2013) Artemin, a Glial Cell Line-Derived Neurotrophic Factor Family Member, Induces TRPM8-Dependent Cold Pain. The Journal of neuroscience: the official journal of the Society for Neuroscience 33(30):12543-12552.
24. Andersson D A, Chase H W, & Bevan S (2004) TRPM8 activation by menthol, icilin, and cold is differentially modulated by intracellular pH. The Journal of neuroscience: the official journal of the Society for Neuroscience 24(23):5364-5369.
25. Lewin G R, Rueff A, & Mendell L M (1994) Peripheral and central mechanisms of NGF-induced hyperalgesia. The European journal of neuroscience 6(12):1903-1912.
26. Yu T, et al. (1998) Expression of GDNF family receptor components during development: implications in the mechanisms of interaction. The Journal of neuroscience: the official journal of the Society for Neuroscience 18(12):4684-4696.
27. Poteryaev D, et al. (1999) GDNF triggers a novel ret-independent Src kinase family-coupled signaling via a GPI-linked GDNF receptor alphal. FEBS letters 463(1-2):63-66.
28. Trupp M, Scott R, Whittemore S R, & Ibanez C F (1999) Ret-dependent and -independent mechanisms of glial cell line-derived neurotrophic factor signaling in neuronal cells. J Biol Chem 274(30):20885-20894.
29. Schmutzler B S, Roy S, Pittman S K, Meadows R M, & Hingtgen C M (2011) Ret-dependent and Ret-independent mechanisms of Gfl-induced sensitization. Molecular pain 7:22.
30. Malin S A, et al. (2006) Glial cell line-derived neurotrophic factor family members sensitize nociceptors in vitro and produce thermal hyperalgesia in vivo. The Journal of neuroscience: the official journal of the Society for Neuroscience 26(33):8588-8599.
31. Thornton P, et al. (2013) Artemin-GFRalpha3 interactions partially contribute to acute inflammatory hypersensitivity. Neurosci Lett 545:23-28.
32. Ikeda-Miyagawa Y, et al. (2015) Peripherally increased artemin is a key regulator of TRPA1/V1 expression in primary afferent neurons. Molecular pain 11:8.
33. Yoshida N, et al. (2011) Inhibition of TRPA1 channel activity in sensory neurons by the glial cell line-derived neurotrophic factor family member, artemin. Molecular pain 7:41.
34. Nishino J, et al. (1999) GFR alpha3, a component of the artemin receptor, is required for migration and survival of the superior cervical ganglion. Neuron 23(4):725-736.
35. Honma Y, et al. (2002) Artemin is a vascular-derived neurotropic factor for developing sympathetic neurons. Neuron 35(2):267-282.
36. Carmillo P, et al. (2005) Glial cell line-derived neurotrophic factor (GDNF) receptor alpha-1 (GFR alpha 1) is highly selective for GDNF versus artemin. Biochemistry 44(7):2545-2554.
37. Baloh R H, et al. (1998) Artemin, a novel member of the GDNF ligand family, supports peripheral and central neurons and signals through the GFRalpha3-RET receptor complex. Neuron 21(6):1291-1302.
38. Bennett G J & Xie Y K (1988) A peripheral mononeuropathy in rat that produces disorders of pain sensation like those seen in man. Pain 33(1):87-107.
39. Ventzel L, et al. (2015) Assessment of acute oxaliplatin-induced cold allodynia: a pilot study. Acta neurologica Scandinavica.
40. Elitt C M, et al. (2006) Artemin overexpression in skin enhances expression of TRPV1 and TRPA1 in cutaneous sensory neurons and leads to behavioral sensitivity to heat and cold. The Journal of neuroscience: the official journal of the Society for Neuroscience 26(33):8578-8587.
41. McKemy D D (2013) The molecular and cellular basis of cold sensation. ACS Chem Neurosci 4(2):238-247.
42. Orozco O E, Walus L, Sah D W, Pepinsky R B, & Sanicola M (2001) GFRalpha3 is expressed predominantly in nociceptive sensory neurons. The European journal of neuroscience 13(11):2177-2182.
43. DeBerry J J, Saloman J L, Dragoo B K, Albers K M, & Davis B M (2015) Artemin Immunotherapy Is Effective in Preventing and Reversing Cystitis-Induced Bladder Hyperalgesia via TRPA1 Regulation. The journal of pain: official journal of the American Pain Society 16(7):628-636.
44. Shinoda M, et al. (2015) Involvement of peripheral artemin signaling in tongue pain: possible mechanism in burning mouth syndrome. Pain.
45. Chuang H H, et al. (2001) Bradykinin and nerve growth factor release the capsaicin receptor from PtdIns(4,5)P2-mediated inhibition. Nature 411(6840):957-962.
46. Prescott E D & Julius D (2003) A modular PIP2 binding site as a determinant of capsaicin receptor sensitivity. Science 300(5623):1284-1288.
47. Woolf C J, Ma Q P, Allchorne A, & Poole S (1996) Peripheral cell types contributing to the hyperalgesic action of nerve growth factor in inflammation. The Journal of neuroscience: the official journal of the Society for Neuroscience 16(8):2716-2723.
48. Bennett G, al-Rashed S, Hoult J R, & Brain S D (1998) Nerve growth factor induced hyperalgesia in the rat hind paw is dependent on circulating neutrophils. Pain 77(3):315-322.
49. Bennett D L (2001) Neurotrophic factors: important regulators of nociceptive function. The Neuroscientist: a review journal bringing neurobiology, neurology and psychiatry 7(1):13-17.
50. Murota H, et al. (2012) Artemin causes hypersensitivity to warm sensation, mimicking warmth-provoked pruritus in atopic dermatitis. The Journal of allergy and clinical immunology 130(3):671-682 e674.
51. Weinkauf B, et al. (2012) Local gene expression changes after U V-irradiation of human skin. PLoS One 7(6):e39411.
52. Osikowicz M, Longo G, Allard S, Cuello A C, & Ribeiro-da-Silva A (2013) Inhibition of endogenous NGF degradation induces mechanical allodynia and thermal hyperalgesia in rats. Molecular pain 9:37.
53. Elitt C M, Malin S A, Koerber H R, Davis B M, & Albers K M (2008) Overexpression of artemin in the tongue increases expression of TRPV1 and TRPA1 in trigeminal afferents and causes oral sensitivity to capsaicin and mustard oil. Brain research 1230:80-90.
54. Jankowski M P, et al. (2009) Sensitization of cutaneous nociceptors after nerve transection and regeneration: possible role of target-derived neurotrophic factor signaling. The Journal of neuroscience: the official journal of the Society for Neuroscience 29(6):1636-1647.
55. Devesa I & Ferrer-Montiel A (2014) Neurotrophins, endocannabinoids and thermo-transient receptor potential: a threesome in pain signalling. The European journal of neuroscience 39(3):353-362.
56. Airaksinen M S & Saarma M (2002) The GDNF family: signalling, biological functions and therapeutic value. Nat Rev Neurosci 3(5):383-394.
57. Paratcha G, Ledda F, & Ibanez C F (2003) The neural cell adhesion molecule NCAM is an alternative signaling receptor for GDNF family ligands. Cell 113(7):867-879.
58. Cao J P, et al. (2008) Integrin beta1 is involved in the signaling of glial cell line-derived neurotrophic factor. J Comp Neurol 509(2):203-210.
59. Jin X, et al. (2004) Modulation of TRPV1 by nonreceptor tyrosine kinase, c-Src kinase. Am J Physiol Cell Physiol 287(2):C558-563.
60. Zhuang Z Y, Xu H, Clapham D E, & Ji R R (2004) Phosphatidylinositol 3-kinase activates ERK in primary sensory neurons and mediates inflammatory heat hyperalgesia through TRPV1 sensitization. The Journal of neuroscience: the official journal of the Society for Neuroscience 24(38):8300-8309.
61. Ji R R, Samad T A, Jin S X, Schmoll R, & Woolf C J (2002) p38 MAPK activation by NGF in primary sensory neurons after inflammation increases TRPV1 levels and maintains heat hyperalgesia. Neuron 36(1):57-68.
62. Zhu W & Oxford G S (2007) Phosphoinositide-3-kinase and mitogen activated protein kinase signaling pathways mediate acute NGF sensitization of TRPV1. Mol Cell Neurosci 34(4):689-700.
63. Bonnington J K & McNaughton P A (2003) Signalling pathways involved in the sensitisation of mouse nociceptive neurones by nerve growth factor. J Physiol 551(Pt 2):433-446.
64. Gauchan P, Andoh T, Kato A, & Kuraishi Y (2009) Involvement of increased expression of transient receptor potential melastatin 8 in oxaliplatin-induced cold allodynia in mice. Neurosci Lett 458(2):93-95.
65. Descoeur J, et al. (2011) Oxaliplatin-induced cold hypersensitivity is due to remodeling of ion channel expression in nociceptors. EMBO Mol Med 3(5):266-278.
66. Morenilla-Palao C, et al. (2014) Ion channel profile of TRPM8 cold receptors reveals a role of TASK-3 potassium channels in thermosensation. Cell Rep 8(5):1571-1582.
67. Deuis J R, et al. (2013) An animal model of oxaliplatin-induced cold allodynia reveals a crucial role for Nav1.6 in peripheral pain pathways. Pain 154(9):1749-1757.
68. Bannwarth B & Kostine M (2014) Targeting nerve growth factor (NGF) for pain management: what does the future hold for NGF antagonists? Drugs 74(6):619-626.
69. Mantyh P W, Koltzenburg M, Mendell L M, Tive L, & Shelton D L (2011) Antagonism of nerve growth factor-TrkA signaling and the relief of pain. Anesthesiology 115(1):189-204.
70. Brenner D S, Golden J P, & Gereau R Wt (2012) A novel behavioral assay for measuring cold sensation in mice. PLoS One 7(6):e39765.
71. Takashima Y, Ma L, & McKemy D D (2010) The development of peripheral cold neural circuits based on TRPM8 expression. Neuroscience 169(2):828-842.

NCBI Reference Sequence: NP_001487.2

| | | | |
|---|---|---|---|
| LOCUS | NP_001487 | 400 aa | linear PRI 15-MAR-2015 |

DEFINITION GDNF family receptor alpha-3 preproprotein [Homo
sapiens].
ACCESSION NP_001487
VERSION NP_001487.2 GI:22035694
DBSOURCE REFSEQ: accession NM_001496.3
KEYWORDS RefSeq.
SOURCE Homo sapiens (human)
ORGANISM Homo sapiens

Eukaryota; Metazoa; Chordata; Craniata; Vertebrata;
Euteleostomi; Mammalia; Eutheria; Euarchontoglires;
Primates; Haplorrhini; Catarrhini; Hominidae; Homo.

REFERENCE 1 (residues 1 to 400)
PUBMED 23351331

| | |
|---|---|
| FEATURES | Location/Qualifiers |

| | |
|---|---|
| source | 1..400 |

/organism=“Homo sapiens”
/db_xref=“taxon:9606”
/chromosome=“5”
/map=“5q31.1-q31.3”

| | |
|---|---|
| Protein | 1..400 |

/product=“GDNF family receptor alpha-3 preproprotein”
/note=“GPI-linked receptor; glial cell line-derived
neurotrophic factor receptor alpha-3; GDNF family receptor
alpha-3; GFR-alpha-3; GDNFR-alpha-3; GDNF receptor
alpha-3”
/calculated_mol_wt=41233

| | |
|---|---|
| sig_peptide | 1..31 |

/inference=“COORDINATES: ab initio
prediction:SignalP:4.0”
/calculated_mol_wt=3296

| | |
|---|---|
| Region | 44..124 |

/region_name=“GDNF”
/note=“GDNF/GAS1 domain; pfam02351”
/db_xref=“CDD:251240”

| | |
|---|---|
| Region | 162..239 |

/region_name=“GDNF”
/note=“GDNF/GAS1 domain; pfam02351”
/db_xref=“CDD:251240”

| | |
|---|---|
| Region | 248..340 |

/region_name=“GDNF”
/note=“GDNF/GAS1 domain; pfam02351”
/db_xref=“CDD:251240”

| | |
|---|---|
| CDS | 1..400 |

/gene=“GFRA3”
/gene_synonym=“GDNFR3”
/coded_by=“NM_001496.3:141..1343”
/db_xref=“CCDS:CCDS4201.1”
/db_xref=“GeneID:2676”
/db_xref=“HGNC:HGNC:4245”
/db_xref=“HPRD:10420”
/db_xref=“MIM:605710”

| ORIGIN | |
|---|---|
| 1 | MVRPLNPRPL PPVVLMLLLL LPPSPLPLAA GDPLPTESRL MNSCLQARRK CQADPTCSAA |
| 61 | YHEILDSCTSS ISTPLPSEEP SVPADCLEAA QQLRNSSLIG CMCHRRMKNQ ACLDIYWTV |
| 121 | HRARSLGNYE LDVSPYEDTV TSKPWKMNLS KLNMLKPDSD LCLKFAMLCT LNDKCDRLRK |
| 181 | AYGEACSGPH CQRHVCLRQL LTFFEKAAEP HAQGLLLCPC APNDRGCGER RRNTIAPNCA |
| 241 | LPPVAPNCLE LRRLCFSDPL CRSRLVDFQT HCHPMDILGT CATEQSRCLR AYLGLIGTAM |
| 301 | TPNFVSNVNT SVALSCTCRG SGNLQEECEM LEGFFSHNPC LTEAIAAKMR FHSQLFSQDW |
| 361 | PHPTFAVMAH QNENPAVRPQ PWVPSLFSCT LPLILLLSLW |

NCBI Reference Sequence: NP_034410.3

| | | | |
|---|---|---|---|
| LOCUS | NP_034410 | 397 aa | linear ROD 26-OCT-2015 |

DEFINITION GDNF family receptor alpha-3 preproprotein [Mus
musculus].
ACCESSION NP_034410 XP_913024
VERSION NP_034410.3 GI:160298126
DBSOURCE REFSEQ: accession NM_010280.4
KEYWORDS RefSeq.
SOURCE Mus musculus (house mouse)
ORGANISM Mus musculus

Eukaryota; Metazoa; Chordata; Craniata; Vertebrata;
Euteleostomi; Mammalia; Eutheria; Euarchontoglires;
Glires; Rodentia; Sciurognathi; Muroidea; Muridae;
Murinae; Mus; Mus.

REFERENCE 1 (residues 1 to 397)
AUTHORS Wong LE, Gibson ME, Arnold HM, Pepinsky B and Frank E.
TITLE Artemin promotes functional long-distance axonal regeneration to
the brainstem after dorsal root crush
JOURNAL Proc. Natl. Acad. Sci. U.S.A. 112 (19), 6170-6175 (2015)
PUBMED 25918373

##Evidence-Data-START##

| Transcript exon combination :: BC066202.1, AK046542.1 | |
|---|---|
| [ECO:0000332] | |
| RNAseq introns | :: mixed/partial sample |

support

SAMN00849374, SAMN00849375
[ECO:0000350]

##Evidence-Data-END##

| | |
|---|---|
| FEATURES | Location/Qualifiers |

| | |
|---|---|
| source | 1..397 |

/organism=“Mus musculus”
/strain=“C57BL/6”
/db_xref=“taxon:10090”
/chromosome=“18”
/map=“18”

| | |
|---|---|
| Protein | 1..397 |

/product=“GDNF family receptor alpha-3 preproprotein”
/note=“GDNF family receptor alpha-3; GDNFR-alpha-3;
GFR alpha-3”

| | |
|---|---|
| sig_peptide | 1..28 |

/inference=“COORDINATES: ab initio
prediction:SignalP:4.0”
/calculated_mol_wt=3045

| | |
|---|---|
| mat_peptide | 29..371 |

/product=“GDNF family receptor alpha-3”
/experiment=“experimental evidence, no additional details
recorded”
/note=“propagated from UniProtKB/Swiss-Prot
(O35118.1)”
/calculated_mol_wt=38286

| | |
|---|---|
| Region | 41..118 |

/region_name=“GDNF”
/note=“GDNF/GAS1 domain; pfam02351”
/db_xref=“CDD:251240”

| | |
|---|---|
| Region | 159..236 |

/region_name=“GDNF”
/note=“GDNF/GAS1 domain; smart00907”
/db_xref=“CDD:197975”

| | |
|---|---|
| Region | 245..337 |

/region_name=“GDNF”
/note=“GDNF/GAS1 domain; pfam02351”
/db_xref=“CDD:251240”

| | |
|---|---|
| CDS | 1..397 |

/gene=“Gfra3”
/gene_synonym=“GFRalpha3; Y15110”
/coded_by=“NM_010280.4:168..1361”
/db_xref=“CCDS:CCDS29133.1”
/db_xref=“GeneID:14587”
/db_xref=“MGI:MGI:1201403”

| ORIGIN | |
|---|---|
| 1 | MGLSWSPRPP LLMILLLVLS LWLPLGAGNS LATENRFVNS CTQARKKCEA NPACKAAYQH |
| 61 | LGSCTSSLSR PLPLEESAMS ADCLEAAEQL RNSSLIDCRC HRRMKHQATC LDIYWTVHPA |
| 121 | RSLGDYELDV SPYEDTVTSK PWKMNLSKLN MLKPDSDLCL KFAMLCTLHD KCDRLRKAYG |
| 181 | EACSGIRCQR HLCLAQLRSF FEKAAESHAQ GLLLCPCAPE DAGCGERRRN TIAPSCALPS |
| 241 | VTPNCLDLRS FCRADPLCRS RLMDFQTHCH PMDILGTCAT EQSRCLRAYL GLIGTAMTPN |
| 301 | FISKVNTTVA LSCTCRGSGN LQDECEQLER SFSQNPCLVE AIAAKMRFHR QLFSQDWADS |
| 361 | TFSVVQQQNS NPALRLQPRL PILSFSILPL ILLQTLW |

| | |
|---|---|
| LOCUS | NP_001129687 228 aa linear |

PRI 15-MAR-2015
DEFINITION artemin isoform 3 precursor [Homo sapiens].
ACCESSION NP_001129687
VERSION NP_001129687.1 GI:209977029
DBSOURCE REFSEQ: accession NM_001136215.1
KEYWORDS RefSeq.
SOURCE Homo sapiens (human)
ORGANISM Homo sapiens

Eukaryota; Metazoa; Chordata; Craniata; Vertebrata;

Euteleostomi; Mammalia; Eutheria; Euarchontoglires; Primates;
Haplorrhini; Catarrhini; Hominidae; Homo.
REFERENCE 1 (residues 1 to 228)
AUTHORS Ding K, Banerjee A, Tan S, Zhao J, Zhuang Q, Li R, Qian P,
Liu S,

Wu ZS, Lobie PE and Zhu T.

TITLE Artemin, a member of the glial cell line-derived neurotrophic
factor family of ligands, is HER2-regulated and mediates acquired
trastuzumab resistance by promoting cancer stem cell-like behavior
in mammary carcinoma cells
JOURNAL J. Biol. Chem. 289 (23), 16057-16071 (2014)
PUBMED 24737320
REMARK GeneRIF: conclude that ARTN is one mediator of acquired
resistance to trastuzumab in HER2-positive mammary carcinoma cells

##Evidence-Data-START##
Transcript exon combination :: BC062375.1 [ECO:0000332]
RNAseq introns:: single sample supports all introns
SAMEA962338 [ECO:0000348]
##Evidence-Data-END##

| | |
|---|---|
| FEATURES | Location/Qualifiers |

| | |
|---|---|
| source | 1..228 |

/organism=“Homo sapiens”
/db_xref=“taxon:9606”
/chromosome=“1”
/map=“1p34.1”

| | |
|---|---|
| Protein | 1..228 |

/product=“artemin isoform 3 precursor”
/note=“neublastin; neurotrophic factor”

| | |
|---|---|
| sig_peptide | 1..47 |

/calculated_mol_wt=4825

| | |
|---|---|
| mat_peptide | 48..228 |

/product=“artemin isoform 3”
/calculated_mol_wt=18810

| | |
|---|---|
| Region | 128..226 |

/region_name=“TGF_beta”
/note=“Transforming growth factor beta like domain;
cl02510”
/db_xref=“CDD:261316”

| | |
|---|---|
| CDS | 1..228 |

/gene=“ARTN”
/gene_synonym=“ENOVIN; EVN; NBN”
/coded_by=“NM_001136215.1:634..1320”
/note=“isoform 3 precursor is encoded by transcript
variant 5”
/db_xref=“CCDS:CCDS502.1”
/db_xref=“GeneID:9048”
/db_xref=“HGNC:HGNC:727”
/db_xref=“MIM:603886”

| ORIGIN | |
|---|---|
| 1 | MELGLGGLST LSHCPWPRQQ APLGLSAQPA LWPTLAALAL LSSVAEASLG SAPRSPAPRE |
| 61 | GPPPVLASPA GHLPGGRTAR WCSGRARRPP PQPSRPAPPP PAPPSALPRG GRAARAGGPG |
| 121 | SRARAAGARG CRLRSQLVPV RALGLGHRSD ELVRFRFCSG SCRRARSPHD LSLASLLGAG |
| 181 | ALRPPPGSRP VSQPCCRPTR YEAVSFMDVN STWRTVDRLS ATACGCLG |

NCBI Reference Sequence: NP_001271120.1

| | | | |
|---|---|---|---|
| LOCUS | NP_001271120 | 224 aa | linear ROD |

15-FEB-2015
DEFINITION artemin isoform 1 precursor [Mus musculus].
ACCESSION NP_001271120
VERSION NP_001271120.1 GI:545746298
DBSOURCE REFSEQ: accession NM_001284191.1
KEYWORDS RefSeq.
SOURCE Mus musculus (house mouse)
ORGANISM Mus musculus

Eukaryota; Metazoa; Chordata; Craniata; Vertebrata;
Euteleostomi; Mammalia; Eutheria; Euarchontoglires;
Glires; Rodentia; Sciurognathi; Muroidea; Muridae;
Murinae; Mus; Mus.

REFERENCE 1 (residues 1 to 224)
AUTHORS Uesaka T, Nagashimada M and Enomoto H.
TITLE GDNF signaling levels control migration and neuronal
differentiation of enteric ganglion precursors
JOURNAL J. Neurosci. 33 (41), 16372-16382 (2013)
PUBMED 24107967

| ##Evidence-Data-START## | |
|---|---|
| Transcript exon combination :: AK015393.1, BY714945.1 | |
| [ECO:0000332] | |
| RNAseq introns | :: single sample supports all introns |

SAMN00849387, SAMN01164131
[ECO:0000348]

##Evidence-Data-END##

| | |
|---|---|
| FEATURES | Location/Qualifiers |

| | |
|---|---|
| source | 1..224 |

/organism=“Mus musculus”
/strain=“C57BL/6”
/db_xref=“taxon:10090”
/chromosome=“4”
/map=“4”

| | |
|---|---|
| Protein | 1..224 |

/product=“artemin isoform 1 precursor”

| | |
|---|---|
| sig_peptide | 1..39 |

/inference=“COORDINATES: ab initio
prediction:SignalP:4.0”
/calculated_mol_wt=4322

| | |
|---|---|
| mat_peptide | 112..224 |

/product=“Artemin”
/experiment=“experimental evidence, no additional details
recorded”
/note=“propagated from UniProtKB/Swiss-Prot
(Q9Z0L2.1)”
/calculated_mol_wt=12108

| | |
|---|---|
| Region | 124..222 |

/region_name=“TGF_beta”
/note=“Transforming growth factor beta like domain;
cl02510”
/db_xref=“CDD:261316”

| | |
|---|---|
| CDS | 1..224 |

/gene=“Artn”
/gene_synonym=“neublastin”
/coded_by=“NM_001284191.1:730..1404”
/note=“isoform 1 precursor is encoded by transcript
variant 1”
/db_xref=“CCDS:CCDS18543.1”
/db_xref=“GeneID:11876”
/db_xref=“MGI:MGI:1333791”

| ORIGIN | |
|---|---|
| 1 | MELGLAEPTA LSHCLRPRWQ SAWWPTLAVL ALLSCVTEAS LDPMSRSPAA RDGPSPVLAP |
| 61 | PTDHLPGGHT AHLCSERTLR PPPQSPQPAP PPPGPALQSP PAALRGARAA RAGTRSSRAR |
| 121 | TTDARGCRLR SQLVPVSALG LGHSSDELIR FRFCSGSCRR ARSQHDLSLA SLLGAGALRS |
| 181 | PPGSRPISQP CCRPTRYEAV SFMDVNSTWR TVDHLSATAC GCLG |

Human NGF (Reproduced from UniProtKB-P001138 (NGF_Human)
MSMLFYTLIT AFLIGIQAEP HSESNVPAGH TIPQAHWTKL QHSLDTALRR
ARSAPAAAIA ARVAGQTRNI TVDPRLFKKR RLRSPRVLFS TQPPREAADT
QDLDFEVGGA APFNRTHRSK RS SSHPIFHR GEFSVCDSVS VWVGDKTTAT
DIKGKEVMVL GEVNINNSVF KQYFFETKCR DPNPVDSGCR GIDSKHWNSY
CTTTHTFVKA LTMDGKQAAW RFIRIDTACV CVLSRKAVRR A

What is claimed is:

1. A method to treat and/or inhibit, and/or reduce pre-existing cold pain and/or pain that induced by NGF in a subject in need thereof, comprising administering to the subject of an effective amount of an agent that interferes with GFRα3 signaling in a cell expressing the GFRα3 receptor.

2. The method of claim 1, wherein the agent interferes with artemin-GFRα3 signaling in the cell.

3. The method of claim 1, wherein the agent is an artemin neutralizing antibody or a fragment, derivative or variant of the artemin neutralizing antibody.

4. The method of claim 3, wherein the antibody comprises the CDR of monoclonal antibody 1085 (mAb 1085), or an equivalent of the CDR of mAb 1085.

5. The method of claim 3, wherein the antibody is a human or an humanized antibody.

6. The method of claim 1, wherein the subject is an animal.

7. A method to treat and/or inhibit, and/or reduce pre-existing cold pain and/or pain that induced by NGF in a subject in need thereof, comprising administering to the subject of an effective amount of an agent that interferes with NGF receptor binding.

8. The method of claim 7, wherein the agent is an NGF neutralizing antibody or a fragment, derivative or variant of the artemin neutralizing antibody.

9. The method of claim 7, wherein the subject is an animal.

10. A method for the treatment of a disease or condition that relates to GFRα3 receptor signaling in a cell or tissue in a subject in need thereof, comprising administering to the subject an effective amount of an agent that interferes with GFRα3 signaling in the cell or the tissue expressing the GFRα3 receptor.

11. The method of claim 10, wherein the agent interferes with artemin-GFRα3 signaling in the cell.

12. The method of claim 10, wherein the agent is an artemin neutralizing antibody or a fragment, derivative or variant of the artemin neutralizing antibody.

13. The method of claim 12, wherein the antibody comprises the CDR of monoclonal antibody 1085 (mAb 1085), or an equivalent of the CDR of mAb 1085.

14. The method of claim 12, wherein the antibody is a human or an humanized antibody.

15. The method of claim 10, wherein the subject is an animal.

16. The method of claim 10, wherein the subject is a human.

17. A method to identify a therapeutic agent to treat and/or inhibit, and/or reduce pre-existing cold pain and/or that induced by NGF in a subject in need thereof comprising contacting a cell that expresses an GFRα3 receptor with a test agent and assaying for interference with GFRα3 signaling in the cell, wherein an agent that interferes with GFRα3 receptor signaling is a potential therapeutic agent and an agent that does not interfere with GFRα3 receptor signaling is not a potential therapeutic agent.

18. The method of claim 17, wherein the cell is an animal or mammalian cell.

19. The method of claim 17, wherein the cell is a human cell.
